SEQUENCE LISTING (141 sequences)

SEQ ID NO: 1 - DNA, 20 bp, Artificial Sequence
  Artificially synthesized sgRNA (control)
gggcgaggagctgttcaccg

SEQ ID NO: 2 - DNA, 20 bp, Artificial Sequence
  Artificially synthesized sgRNA (control)
gagctggacggcgacgtaaa

SEQ ID NO: 3 - DNA, 20 bp, Artificial Sequence
  Artificially synthesized sgRNA for CASP8AP2
gatgccagggagacctcggt

SEQ ID NO: 4 - DNA, 21 bp, Artificial Sequence
  Artificially synthesized sgRNA for CASP8AP2
gctgcccggcccaagacaacc

SEQ ID NO: 5 - DNA, 21 bp, Artificial Sequence
  Artificially synthesized sgRNA for CASP8AP2
gcggttcctttctgcccaccg

SEQ ID NO: 6 - DNA, 20 bp, Artificial Sequence
  Artificially synthesized oligonucleotide primer
atcatggcagcagatgatga

SEQ ID NO: 7 - DNA, 20 bp, Artificial Sequence
  Artificially synthesized oligonucleotide primer
cccagcgtatatgtccagtg

SEQ ID NO: 8 - DNA, 19 bp, Artificial Sequence
  Artificially synthesized oligonucleotide primer
gtgaaggtcggagtcaacg

SEQ ID NO: 9 - DNA, 19 bp, Artificial Sequence
  Artificially synthesized oligonucleotide primer
tgaggtcaatgaaggggtc

SEQ ID NO: 10 - DNA, 22 bp, Artificial Sequence
  Artificially synthesized oligonucleotide primer
aggtggacctggagactctcag

SEQ ID NO: 11 - DNA, 22 bp, Artificial Sequence
  Artificially synthesized oligonucleotide primer
tcctcttggagaagatcagccg

SEQ ID NO: 12 - DNA, 20 bp, Artificial Sequence
  Artificially synthesized oligonucleotide primer
ggctcttcgtgtgaggattc

SEQ ID NO: 13 - DNA, 23 bp, Artificial Sequence
  Artificially synthesized oligonucleotide primer
ctgggaggtagaggttgtaatga

SEQ ID NO: 14 - DNA, 18 bp, Artificial Sequence
  Artificially synthesized oligonucleotide primer
tttccagagctccgctgt

SEQ ID NO: 15 - DNA, 21 bp, Artificial Sequence
  Artificially synthesized oligonucleotide primer
tgtcctcaaatagccctacca

SEQ ID NO: 16 - DNA, 18 bp, Artificial Sequence
  Artificially synthesized oligonucleotide primer
tccagggagatccagacg

SEQ ID NO: 17 - DNA, 19 bp, Artificial Sequence
  Artificially synthesized oligonucleotide primer
gtacttggtgacggccttg

SEQ ID NO: 18 - DNA, 18 bp, Artificial Sequence
  Artificially synthesized oligonucleotide primer
accaaggctgctcgaaag

SEQ ID NO: 19 - DNA, 18 bp, Artificial Sequence
  Artificially synthesized oligonucleotide primer
ggtaacggtggggcttct

SEQ ID NO: 20 - DNA, 19 bp, Artificial Sequence
  Artificially synthesized oligonucleotide siPool1, siRNA1, sense
gaaacagacttaactaata

SEQ ID NO: 21 - DNA, 19 bp, Artificial Sequence
  Artificially synthesized oligonucleotide siPool1, siRNA1, antisense
tattagttaagtctgtttc

SEQ ID NO: 22 - DNA, 19 bp, Artificial Sequence
  Artificially synthesized oligonucleotide siPool1, siRNA2, sense
gcaaggttctgatctttta

SEQ ID NO: 23 - DNA, 19 bp, Artificial Sequence
  Artificially synthesized oligonucleotide siPool1, siRNA2, antisense
taaaagatcagaaccttgc

SEQ ID NO: 24 - DNA, 19 bp, Artificial Sequence
  Artificially synthesized oligonucleotide siPool1, siRNA3, sense
gtgactaccttacagaaga

SEQ ID NO: 25 - DNA, 19 bp, Artificial Sequence
  Artificially synthesized oligonucleotide siPool1, siRNA3, antisense
tcttctgtaaggtagtcac

SEQ ID NO: 26 - DNA, 19 bp, Artificial Sequence
  Artificially synthesized oligonucleotide siPool1, siRNA4, sense
gtgatgtattgaaatctta

SEQ ID NO: 27 - DNA, 19 bp, Artificial Sequence
  Artificially synthesized oligonucleotide siPool1, siRNA4, antisense
taagatttcaatacatcac

SEQ ID NO: 28 - DNA, 19 bp, Artificial Sequence
  Artificially synthesized oligonucleotide siPool1, siRNA5, sense
gtgatgaagtggctgatga

SEQ ID NO: 29 - DNA, 19 bp, Artificial Sequence
  Artificially synthesized oligonucleotide siPool1, siRNA5, antisense
tcatcagccacttcatcac

SEQ ID NO: 30 - DNA, 19 bp, Artificial Sequence
  Artificially synthesized oligonucleotide siPool1, siRNA6, sense
gttcgaagtttaaaagtaa

SEQ ID NO: 31 - DNA, 19 bp, Artificial Sequence
  Artificially synthesized oligonucleotide siPool1, siRNA6, antisense
ttacttttaaacttcgaac

SEQ ID NO: 32 - DNA, 19 bp, Artificial Sequence
  Artificially synthesized oligonucleotide siPool1, siRNA7, sense
gtgatattatagagtctaa

SEQ ID NO: 33 - DNA, 19 bp, Artificial Sequence
  Artificially synthesized oligonucleotide siPool1, siRNA7, antisense
ttagactctataatatcac

SEQ ID NO: 34 - DNA, 19 bp, Artificial Sequence
  Artificially synthesized oligonucleotide siPool1, siRNA8, sense
gttttggtatattgtaaaa

SEQ ID NO: 35 - DNA, 19 bp, Artificial Sequence
  Artificially synthesized oligonucleotide siPool1, siRNA8, antisense
ttttacaatataccaaaac

SEQ ID NO: 36 - DNA, 19 bp, Artificial Sequence
  Artificially synthesized oligonucleotide siPool1, siRNA9, sense
gagaattgctgaaagaaaa

SEQ ID NO: 37 - DNA, 19 bp, Artificial Sequence
  Artificially synthesized oligonucleotide siPool1, siRNA9, antisense
ttttctttcagcaattctc

SEQ ID NO: 38 - DNA, 19 bp, Artificial Sequence
  Artificially synthesized oligonucleotide siPool1, siRNA10, sense
gacatttgatacagttaaa

SEQ ID NO: 39 - DNA, 19 bp, Artificial Sequence
  Artificially synthesized oligonucleotide siPool1, siRNA10, antisense
tttaactgtatcaaatgtc

SEQ ID NO: 40 - DNA, 19 bp, Artificial Sequence
  Artificially synthesized oligonucleotide siPool1, siRNA11, sense
gcttggaattggataccaa

SEQ ID NO: 41 - DNA, 19 bp, Artificial Sequence
  Artificially synthesized oligonucleotide siPool1, siRNA11, antisense
ttggtatccaattccaagc

SEQ ID NO: 42 - DNA, 19 bp, Artificial Sequence
  Artificially synthesized oligonucleotide siPool1, siRNA12, sense
gtgggaaaatacaccttta

SEQ ID NO: 43 - DNA, 19 bp, Artificial Sequence
  Artificially synthesized oligonucleotide siPool1, siRNA12, antisense
taaaggtgtattttcccac

SEQ ID NO: 44 - DNA, 19 bp, Artificial Sequence
  Artificially synthesized oligonucleotide siPool1, siRNA13, sense
gaaaatagtttgttagtta

SEQ ID NO: 45 - DNA, 19 bp, Artificial Sequence
  Artificially synthesized oligonucleotide siPool1, siRNA13, antisense
taactaacaaactattttc

SEQ ID NO: 46 - DNA, 19 bp, Artificial Sequence
  Artificially synthesized oligonucleotide siPool1, siRNA14, sense
ggctttcagtaccacccta

SEQ ID NO: 47 - DNA, 19 bp, Artificial Sequence
  Artificially synthesized oligonucleotide siPool1, siRNA14, antisense
tagggtggtactgaaagcc

SEQ ID NO: 48 - DNA, 19 bp, Artificial Sequence
  Artificially synthesized oligonucleotide siPool1, siRNA15, sense
gtcctaaaatgattagtga

SEQ ID NO: 49 - DNA, 19 bp, Artificial Sequence
  Artificially synthesized oligonucleotide siPool1, siRNA15, antisense
tcactaatcattttaggac

SEQ ID NO: 50 - DNA, 19 bp, Artificial Sequence
  Artificially synthesized oligonucleotide siPool1, siRNA16, sense
gctgatattttgccaataa

SEQ ID NO: 51 - DNA, 19 bp, Artificial Sequence
  Artificially synthesized oligonucleotide siPool1, siRNA16, antisense
ttattggcaaaatatcagc

SEQ ID NO: 52 - DNA, 19 bp, Artificial Sequence
  Artificially synthesized oligonucleotide siPool1, siRNA17, sense
gaaattgtttggacttata

SEQ ID NO: 53 - DNA, 19 bp, Artificial Sequence
  Artificially synthesized oligonucleotide siPool1, siRNA17, antisense
tataagtccaaacaatttc

SEQ ID NO: 54 - DNA, 19 bp, Artificial Sequence
  Artificially synthesized oligonucleotide siPool1, siRNA18, sense
gcagaagactaataaggaa

SEQ ID NO: 55 - DNA, 19 bp, Artificial Sequence
  Artificially synthesized oligonucleotide siPool1, siRNA18, antisense
ttccttattagtcttctgc

SEQ ID NO: 56 - DNA, 19 bp, Artificial Sequence
  Artificially synthesized oligonucleotide siPool1, siRNA19, sense
gacctaaaattgagtttta

SEQ ID NO: 57 - DNA, 19 bp, Artificial Sequence
  Artificially synthesized oligonucleotide siPool1, siRNA19, antisense
taaaactcaattttaggtc

SEQ ID NO: 58 - DNA, 19 bp, Artificial Sequence
  Artificially synthesized oligonucleotide siPool1, siRNA20, sense
ggcatatgttccctccata

SEQ ID NO: 59 - DNA, 19 bp, Artificial Sequence
  Artificially synthesized oligonucleotide siPool1, siRNA20, antisense
tatggagggaacatatgcc

SEQ ID NO: 60 - DNA, 19 bp, Artificial Sequence
  Artificially synthesized oligonucleotide siPool1, siRNA21, sense
ggtcattattatcatctga

SEQ ID NO: 61 - DNA, 19 bp, Artificial Sequence
  Artificially synthesized oligonucleotide siPool1, siRNA21, antisense
tcagatgataataatgacc

SEQ ID NO: 62 - DNA, 19 bp, Artificial Sequence
  Artificially synthesized oligonucleotide siPool1, siRNA22, sense
gcccagacatggaaaggaa

SEQ ID NO: 63 - DNA, 19 bp, Artificial Sequence
  Artificially synthesized oligonucleotide siPool1, siRNA22, antisense
ttcctttccatgtctgggc

SEQ ID NO: 64 - DNA, 19 bp, Artificial Sequence
  Artificially synthesized oligonucleotide siPool1, siRNA23, sense
ggaccaagatcactggaga

SEQ ID NO: 65 - DNA, 19 bp, Artificial Sequence
  Artificially synthesized oligonucleotide siPool1, siRNA23, antisense
tctccagtgatcttggtcc

SEQ ID NO: 66 - DNA, 19 bp, Artificial Sequence
  Artificially synthesized oligonucleotide siPool1, siRNA24, sense
gaaatgactttctagatta

SEQ ID NO: 67 - DNA, 19 bp, Artificial Sequence
  Artificially synthesized oligonucleotide siPool1, siRNA24, antisense
taatctagaaagtcatttc

SEQ ID NO: 68 - DNA, 19 bp, Artificial Sequence
  Artificially synthesized oligonucleotide siPool1, siRNA25, sense
ggaaaattatcaagaccaa

SEQ ID NO: 69 - DNA, 19 bp, Artificial Sequence
  Artificially synthesized oligonucleotide siPool1, siRNA25, antisense
ttggtcttgataattttcc

SEQ ID NO: 70 - DNA, 19 bp, Artificial Sequence
  Artificially synthesized oligonucleotide siPool1, siRNA26, sense
ggctttgcctagccaagga

SEQ ID NO: 71 - DNA, 19 bp, Artificial Sequence
  Artificially synthesized oligonucleotide siPool1, siRNA26, antisense
tccttggctaggcaaagcc

SEQ ID NO: 72 - DNA, 19 bp, Artificial Sequence
  Artificially synthesized oligonucleotide siPool1, siRNA27, sense
ggaaagaagttaaatcaca

SEQ ID NO: 73 - DNA, 19 bp, Artificial Sequence
  Artificially synthesized oligonucleotide siPool1, siRNA27, antisense
tgtgatttaacttctttcc

SEQ ID NO: 74 - DNA, 19 bp, Artificial Sequence
  Artificially synthesized oligonucleotide siPool1, siRNA28, sense
gagtggaaataaaccgcaa

SEQ ID NO: 75 - DNA, 19 bp, Artificial Sequence
  Artificially synthesized oligonucleotide siPool1, siRNA28, antisense
ttgcggtttatttccactc

SEQ ID NO: 76 - DNA, 19 bp, Artificial Sequence
  Artificially synthesized oligonucleotide siPool1, siRNA29, sense
gccctgcgtttcttccata

SEQ ID NO: 77 - DNA, 19 bp, Artificial Sequence
  Artificially synthesized oligonucleotide siPool1, siRNA29, antisense
tatggaagaaacgcagggc

SEQ ID NO: 78 - DNA, 19 bp, Artificial Sequence
  Artificially synthesized oligonucleotide siPool1, siRNA30, sense
gcttctccatgtcctaaaa

SEQ ID NO: 79 - DNA, 19 bp, Artificial Sequence
  Artificially synthesized oligonucleotide siPool1, siRNA30, antisense
ttttaggacatggagaagc

SEQ ID NO: 80 - DNA, 19 bp, Artificial Sequence
  Artificially synthesized oligonucleotide siPool2, siRNA1, sense
ctggagccttgcttaccta

SEQ ID NO: 81 - DNA, 19 bp, Artificial Sequence
  Artificially synthesized oligonucleotide siPool2, siRNA1, antisense
taggtaagcaaggctccag

SEQ ID NO: 82 - DNA, 19 bp, Artificial Sequence
  Artificially synthesized oligonucleotide siPool2, siRNA2, sense
caacagtgatgtattgaaa

SEQ ID NO: 83 - DNA, 19 bp, Artificial Sequence
  Artificially synthesized oligonucleotide siPool2, siRNA2, antisense
tttcaatacatcactgttg

SEQ ID NO: 84 - DNA, 19 bp, Artificial Sequence
  Artificially synthesized oligonucleotide siPool2, siRNA3, sense
ctacaagtctttctgcaaa

SEQ ID NO: 85 - DNA, 19 bp, Artificial Sequence
  Artificially synthesized oligonucleotide siPool2, siRNA3, antisense
tttgcagaaagacttgtag

SEQ ID NO: 86 - DNA, 19 bp, Artificial Sequence
  Artificially synthesized oligonucleotide siPool2, siRNA4, sense
ggctgatgaatgtttcaaa

SEQ ID NO: 87 - DNA, 19 bp, Artificial Sequence
  Artificially synthesized oligonucleotide siPool2, siRNA4, antisense
tttgaaacattcatcagcc

SEQ ID NO: 88 - DNA, 19 bp, Artificial Sequence
  Artificially synthesized oligonucleotide siPool2, siRNA5, sense
catcgagccttacttttaa

SEQ ID NO: 89 - DNA, 19 bp, Artificial Sequence
  Artificially synthesized oligonucleotide siPool2, siRNA5, antisense
ttaaaagtaaggctcgatg

SEQ ID NO: 90 - DNA, 19 bp, Artificial Sequence
  Artificially synthesized oligonucleotide siPool2, siRNA6, sense
cttgattatttgtttgcaa

SEQ ID NO: 91 - DNA, 19 bp, Artificial Sequence
  Artificially synthesized oligonucleotide siPool2, siRNA6, antisense
ttgcaaacaaataatcaag

SEQ ID NO: 92 - DNA, 19 bp, Artificial Sequence
  Artificially synthesized oligonucleotide siPool2, siRNA7, sense
cacaaaatataaataggaa

SEQ ID NO: 93 - DNA, 19 bp, Artificial Sequence
  Artificially synthesized oligonucleotide siPool2, siRNA7, antisense
ttcctatttatattttgtg

SEQ ID NO: 94 - DNA, 19 bp, Artificial Sequence
  Artificially synthesized oligonucleotide siPool2, siRNA8, sense
gagggaggtttatcacctt

SEQ ID NO: 95 - DNA, 19 bp, Artificial Sequence
  Artificially synthesized oligonucleotide siPool2, siRNA8, antisense
aaggtgataaacctccctc

SEQ ID NO: 96 - DNA, 19 bp, Artificial Sequence
  Artificially synthesized oligonucleotide siPool2, siRNA9, sense
catgttagaacttaccaaa

SEQ ID NO: 97 - DNA, 19 bp, Artificial Sequence
  Artificially synthesized oligonucleotide siPool2, siRNA9, antisense
tttggtaagttctaacatg

SEQ ID NO: 98 - DNA, 19 bp, Artificial Sequence
  Artificially synthesized oligonucleotide siPool2, siRNA10, sense
catgttagaacttaccaaa

SEQ ID NO: 99 - DNA, 19 bp, Artificial Sequence
  Artificially synthesized oligonucleotide siPool2, siRNA10, antisense
tttggttccaataacgttg

SEQ ID NO: 100 - DNA, 19 bp, Artificial Sequence
  Artificially synthesized oligonucleotide siPool2, siRNA11, sense
ccctcatcattaaaggcaa

SEQ ID NO: 101 - DNA, 19 bp, Artificial Sequence
  Artificially synthesized oligonucleotide siPool2, siRNA11, antisense
ttgcctttaatgatgaggg

SEQ ID NO: 102 - DNA, 19 bp, Artificial Sequence
  Artificially synthesized oligonucleotide siPool2, siRNA12, sense
gtgctagtcatcattctga

SEQ ID NO: 103 - DNA, 19 bp, Artificial Sequence
  Artificially synthesized oligonucleotide siPool2, siRNA12, antisense
tcagaatgatgactagcac

SEQ ID NO: 104 - DNA, 19 bp, Artificial Sequence
  Artificially synthesized oligonucleotide siPool2, siRNA13, sense
ggaattggataccaaaaga

SEQ ID NO: 105 - DNA, 19 bp, Artificial Sequence
  Artificially synthesized oligonucleotide siPool2, siRNA13, antisense
tcttttggtatccaattcc

SEQ ID NO: 106 - DNA, 19 bp, Artificial Sequence
  Artificially synthesized oligonucleotide siPool2, siRNA14, sense
cagcaagcatcgagcctta

SEQ ID NO: 107 - DNA, 19 bp, Artificial Sequence
  Artificially synthesized oligonucleotide siPool2, siRNA14, antisense
taaggctcgatgcttgctg

SEQ ID NO: 108 - DNA, 19 bp, Artificial Sequence
  Artificially synthesized oligonucleotide siPool2, siRNA15, sense
gatgggtcatagtgatgaa

SEQ ID NO: 109 - DNA, 19 bp, Artificial Sequence
  Artificially synthesized oligonucleotide siPool2, siRNA15, antisense
ttcatcactatgacccatc

SEQ ID NO: 110 - DNA, 19 bp, Artificial Sequence
  Artificially synthesized oligonucleotide siPool2, siRNA16, sense
cagtgactctctaatttca

SEQ ID NO: 111 - DNA, 19 bp, Artificial Sequence
  Artificially synthesized oligonucleotide siPool2, siRNA16, antisense
tgaaattagagagtcactg

SEQ ID NO: 112 - DNA, 19 bp, Artificial Sequence
  Artificially synthesized oligonucleotide siPool2, siRNA17, sense
ctgatttgcttatctgttt

SEQ ID NO: 113 - DNA, 19 bp, Artificial Sequence
  Artificially synthesized oligonucleotide siPool2, siRNA17, antisense
aaacagataagcaaatcag

SEQ ID NO: 114 - DNA, 19 bp, Artificial Sequence
  Artificially synthesized oligonucleotide siPool2, siRNA18, sense
ctgggactcttaactgata

SEQ ID NO: 115 - DNA, 19 bp, Artificial Sequence
  Artificially synthesized oligonucleotide siPool2, siRNA18, antisense
tatcagttaagagtcccag

SEQ ID NO: 116 - DNA, 19 bp, Artificial Sequence
  Artificially synthesized oligonucleotide siPool2, siRNA19, sense
ctcagattctgactataaa

SEQ ID NO: 117 - DNA, 19 bp, Artificial Sequence
  Artificially synthesized oligonucleotide siPool2, siRNA19, antisense
tttatagtcagaatctgag

SEQ ID NO: 118 - DNA, 19 bp, Artificial Sequence
  Artificially synthesized oligonucleotide siPool2, siRNA20, sense
gccatgcatcagtgggaaa

SEQ ID NO: 119 - DNA, 19 bp, Artificial Sequence
  Artificially synthesized oligonucleotide siPool2, siRNA20, antisense
tttcccactgatgcatggc

SEQ ID NO: 120 - DNA, 19 bp, Artificial Sequence
  Artificially synthesized oligonucleotide siPool2, siRNA21, sense
ctgaaggtatggagatgaa

SEQ ID NO: 121 - DNA, 19 bp, Artificial Sequence
  Artificially synthesized oligonucleotide siPool2, siRNA21, antisense
ttcatctccataccttcag

SEQ ID NO: 122 - DNA, 19 bp, Artificial Sequence
  Artificially synthesized oligonucleotide siPool2, siRNA22, sense
gatatgtcgtccagttcaa

SEQ ID NO: 123 - DNA, 19 bp, Artificial Sequence
  Artificially synthesized oligonucleotide siPool2, siRNA22, antisense
ttgaactggacgacatatc

SEQ ID NO: 124 - DNA, 19 bp, Artificial Sequence
  Artificially synthesized oligonucleotide siPool2, siRNA23, sense
caataacagtaatgtataa

SEQ ID NO: 125 - DNA, 19 bp, Artificial Sequence
  Artificially synthesized oligonucleotide siPool2, siRNA23, antisense
ttatacattactgttattg

SEQ ID NO: 126 - DNA, 19 bp, Artificial Sequence
  Artificially synthesized oligonucleotide siPool2, siRNA24, sense
ctccagtattaacactgta

SEQ ID NO: 127 - DNA, 19 bp, Artificial Sequence
  Artificially synthesized oligonucleotide siPool2, siRNA24, antisense
tacagtgttaatactggag

SEQ ID NO: 128 - DNA, 19 bp, Artificial Sequence
  Artificially synthesized oligonucleotide siPool2, siRNA25, sense
gcatgttcagcttgtgcta

SEQ ID NO: 129 - DNA, 19 bp, Artificial Sequence
  Artificially synthesized oligonucleotide siPool2, siRNA25, antisense
tagcacaagctgaacatgc

SEQ ID NO: 130 - DNA, 19 bp, Artificial Sequence
  Artificially synthesized oligonucleotide siPool2, siRNA26, sense
ggctttcattgccggttca

SEQ ID NO: 131 - DNA, 19 bp, Artificial Sequence
  Artificially synthesized oligonucleotide siPool2, siRNA26, antisense
tgaaccggcaatgaaagcc

SEQ ID NO: 132 - DNA, 19 bp, Artificial Sequence
  Artificially synthesized oligonucleotide siPool2, siRNA27, sense
gtataataaaaccatgtaa

SEQ ID NO: 133 - DNA, 19 bp, Artificial Sequence
  Artificially synthesized oligonucleotide siPool2, siRNA27, antisense
ttacatggttttattatac

SEQ ID NO: 134 - DNA, 19 bp, Artificial Sequence
  Artificially synthesized oligonucleotide siPool2, siRNA28, sense
cttagtatgtgattccaaa

SEQ ID NO: 135 - DNA, 19 bp, Artificial Sequence
  Artificially synthesized oligonucleotide siPool2, siRNA28, antisense
tttggaatcacatactaag

SEQ ID NO: 136 - DNA, 19 bp, Artificial Sequence
  Artificially synthesized oligonucleotide siPool2, siRNA29, sense
cactaatggtgtttggtca

SEQ ID NO: 137 - DNA, 19 bp, Artificial Sequence
  Artificially synthesized oligonucleotide siPool2, siRNA29, antisense
tgaccaaacaccattagtg

SEQ ID NO: 138 - DNA, 19 bp, Artificial Sequence
  Artificially synthesized oligonucleotide siPool2, siRNA30, sense
cagaggaactgaaagagta

SEQ ID NO: 139 - DNA, 19 bp, Artificial Sequence
  Artificially synthesized oligonucleotide siPool2, siRNA30, antisense
tactctttcagttcctctg

SEQ ID NO: 140 - protein, 11 aa, Homo sapiens
SMMPDELLTSL

SEQ ID NO: 141 - protein, 9 aa, Homo sapiens
KLDKNPNQV

(19) United States
(12) Patent Application Publication
(10) Pub. No.: US 2024/0002845 A1
(43) Pub. Date: Jan. 4, 2024

(54) CASP8AP2 ANTAGONISTS FOR USE IN THE PREVENTION OR TREATMENT OF CANCER

(71) Applicants: Albert-Ludwigs-Universitat Freiburg, Freiburg im Breisgau (DE); DEUTSCHES KREBSFORSCHUNGSZENTRUM, Heidelberg (DE)

(72) Inventors: Sven Diederichs, Heidelberg (DE); Ksenia Myacheva, Freiburg (DE)

(73) Assignees: Albert-Ludwigs-Universitat Freiburg, Freiburg im Breisgau (DE); DEUTSCHES KREBSFORSCHUNGSZENTRUM, Heidelberg (DE)

(21) Appl. No.: 18/031,001

(22) PCT Filed: Oct. 7, 2021
(86) PCT No.: PCT/EP2021/077761

§ 371 Date: Apr. 9, 2023

(30) Foreign Application Priority Data
Oct. 8, 2020 (EP) 20200810.8

(51) Int. Cl. C12N 15/113; G01N 33/50; A61K 31/7088; A61P 35/00

(52) U.S. Cl. CPC C12N 15/113; G01N 33/5011; A61K 31/7088; C12N 2310/20; C12N 2310/11; C12N 2310/14; A61P 35/00

(57) ABSTRACT

The present invention provides the use of a CASP8AP2 antagonist in the prevention or treatment of cancer. Preferably, the antagonist reduces the viability of the cancer cells without significantly reducing the viability of normal cells in the subject to be treated. Further, methods of treatment and pharmaceutical compositions suitable in said methods are provided, as well.

### TECHNICAL FIELD

The present invention relates to the use of a CASP8AP2 antagonist in the prevention or treatment of cancer. Preferably the antagonist reduces the viability of the cancer cells without significantly reducing the viability of normal cells in the subject to be treated. Further, methods of treatment and pharmaceutical compositions suitable in said methods are provided, as well.

### BACKGROUND OF THE INVENTION

Cancer is the second leading cause of mortality worldwide. Overall, the prevalence of cancer has actually increased. In men, the highest percentage of cancer types occur in the prostate, lung and bronchus, colon and rectum, and urinary bladder. In women, cancer prevalence is highest in the breast, lung and bronchus, colon and rectum, uterine corpus and thyroid (Hassanpour et al., J. Cancer Res. Practice 4 (2017), 127-129).

Lung cancer is the most frequent cause of cancer-related death in the world. Its five-year survival rate is one of the lowest among cancers (3-17%), and it has remained largely unchanged since 1970s despite significant research and clinical advancements. Lung cancer is subdivided into two main histological groups, namely small cell lung cancer (SCLC, 15% of all cases) and non-small cell lung cancer (NSCLC, 85%). Lung adenocarcinoma (LUAD) is a subtype of NSCLC and the most common type of lung cancer, accounting for approx. 40% of all cases.

Surgery, chemo- and radiotherapy are the standard first-line treatments available for early-stage lung cancer. However, approximately 75% of patients are diagnosed with NSCLC at an advanced stage (III/I), and approximately 60-70% of those patients have specific genomic aberrations resulting in potentially actionable molecular targets. Therefore, options for targeted molecular therapies have been actively researched. Approved targeted therapeutics include a number of epidermal growth factor receptor tyrosine kinase antagonists (EGFR TKIs) for patients with EGFR-activating mutations (detected in approximately 20% of LUAD cases) and ALK TKIs for patients with oncogenic ALK gene rearrangements (2-7%).

Despite strong activity and high primary response rates, toxicity and acquired resistance present major challenges for targeted therapy of LUAD.

Hummon et al., Mol. Cancer 11 (2012), 1-22 describes that the cell viability of colon adenocarcinoma cell lines was reduced upon incubation with siCASP8AP2. Hummon et al. did not test the impact of the siCASP8AP2 on the cell viability of untransformed colon cells.

WO 2017/060169 A1 relates to peptides for use in immunotherapy against small cell lung cancer and other cancers. WO' 169 describes inter alia CASP8AP2 peptides comprising the amino acid sequences SMMPDELLTSL and KLDKNPNQV, respectively. WO' 169 does not describe any CASP8AP2 antagonistic activity of these peptides. These CASP8AP2 peptides do not represent CASP8AP2 antagonists in the sense of the present application.

In view thereof, there is a need for the provision of novel means and methods for the treatment of cancer, in particular lung adenocarcinoma. In particular, there is the need for an efficient treatment of cancer which reduces viability of cancer cells but does not significantly adversely affect the viability of non-transformed cells in the patient.

### SUMMARY OF THE INVENTION

The technical problems underlying the invention are solved by the provision of the subject-matter as defined in the claims.

According to a first aspect, the present invention provides a Caspase-8 associated protein-2 (CASP8AP2) antagonist for use in the prevention or treatment of cancer, with the proviso that the CASP8AP2 antagonist is not a peptide comprising the amino acid sequence SMMPDELLTSL (SEQ ID NO: 140) or a peptide comprising the amino acid sequence KLDKNPNQV (SEQ ID NO:141), preferably the cancer is selected from the group consisting of lung cancer, breast cancer, pancreatic cancer or liver cancer.

According to a second aspect, a method for treating cancer comprising administering said CASP8AP2 antagonist to a patient in need thereof is provided.

According to a third aspect, a pharmaceutical composition comprising said CASP8AP2 antagonist and a pharmaceutically acceptable excipient.

According to a fourth aspect, a method for identifying a CASP8AP2 antagonist is provided comprising
i) screening a test compound for binding to CASP8AP2 protein;
ii) optionally determining in vitro the caspase-mediated apoptotic activity induced by the test compound identified in step i) in a cancer cell line, wherein the caspase is selected from caspase-3/7, caspase-8 and caspase-9; and
iii) selecting the test compound as a CASP8AP2 antagonist if the test compound binds to CASP8AP2 and optionally increases the apoptotic activity of Caspase-3/7 and/or Caspase-8 and/or Caspase-9 compared to the caspase-mediated apoptotic activity in the absence of the test compound.

According to a fifth aspect, a method of identifying a CASP8AP2 antagonist is provided comprising
i) screening a test compound for binding to CASP8AP2 protein; and
ii) optionally determining in vitro the cell viability of a cancer cell induced by the test compound identified in step i); and
iii) selecting the test compound as a CASP8AP2 antagonist if the test compound binds to CASP8AP2 and optionally reduces the cell viability of the cancer cell compared to the cell viability of the cancer cell in the absence of the test compound.

According to a sixth aspect, a method for preparing a pharmaceutical composition is provided comprising identifying a CASP8AP2 antagonist by the aforementioned method and mixing the identified CASP8AP2 antagonist with at least one pharmaceutically acceptable excipient.

The inventors surprisingly found that a CASP8AP2 antagonist more strongly and significantly reduces the viability of cancer cells compared to normal, i.e. non-transformed cell lines, that remain largely viable. Thus, the treatment is considered to be efficient and safe.

By using a CRISPRi-dropout screen the inventors could show that CASP8AP2 represents an essential viability factor in lung adenocarcinoma cells. Knockdown of the CASP8AP2 gene in cancer cells, including lung adenocarcinoma, breast cancer, pancreatic cancer and liver cancer reduced their viability to a larger extent than normal (non-transformed) cells from the lung.

FLICE/caspase-8-associated huge protein (FLASH)/CASP8AP2, which was originally identified as a caspase-8-associated protein, is a high molecular weight protein with both a putative nuclear export signal (NES) and nuclear localization signal (NLS). CASP8AP2 was reported to play a role in various cellular processes, including cell cycle progression especially in the S phase, processing of histone mRNA, regulation of apoptosis, and transcriptional control (Minamida et al., PLOS One 9 (2014), e108032, 1-11).

CASP8AP2 was reported to be involved in CD95(Fas)-mediated activation of caspase-8 during apoptosis (Imai et al., Nature 398 (1999), 777-785). CD95 (Fas) is a cell-surface receptor molecule that relays apoptotic (cell death) signals into cells. When Fas is activated by binding of its ligand, the proteolytic protein caspase-8 is recruited to a signalling complex known as death-inducing signalling complex (DISC) by binding to a Fas-associated adapter protein. Stimulated Fas binds CASP8AP2, so CASP8AP2 is probably a component of the DISC signalling complex. In the absence of stimulated Fas CASP8AP2 binds to caspase-8. CASP8AP2 was therefore prior to the present invention considered as necessary for the activation of caspase-8 in Fas-mediated apoptosis.

The present invention is therefore contrary to the assumption in the prior art that CASP8AP2 acts as a general pro-apoptotic factor. It is in particular surprising that the CASP8AP2 antagonist for use according to the invention significantly reduces the viability of the cancer cells compared to the viability of normal cells.

### BRIEF DESCRIPTION OF THE DRAWINGS

FIG. 1 A shows the CRISPRi screen workflow.

FIG. 1 B relates to the robust rank aggregation (RRA) scores for negative selection between all endpoint and reference samples for CASP8AP2, core essential gene transcripts (N=289), positive control gene transcripts (N=127) versus non-targeting sgRNAs aggregated computationally to negative control transcripts (N=205) (Student's t-test; three asterisks represent p<0.001)

FIG. 1 C shows the fold-change in sgRNA-corresponding read counts between all endpoint and reference samples for CASP8AP2, core essential gene transcripts (N=289), positive control gene transcripts (N=127) versus negative control transcripts (N=205) (Student's t-test; three asterisks represent p<0.001)

FIG. 2 A: CellTiter-Glo cell viability assay (CTG) showed a significant decrease in luminescence signal in LUAD cells transduced independently with three constructs expressing sgRNAs against CASP8AP2 (sgC8AP2) compared to two negative control sgRNAs (sgNC) (Student's t-test; three asterisks represent p<0.001; N=3 per condition)

FIG. 2 B: RT-qPCR confirmed significant decrease in CASP8AP2 expression upon knockdown with three independent sgRNAs against CASP8AP2 (sgC8AP2) compared to two negative control sgRNAs (sgNC) (Student's t-test; three asterisks represent p<0.001; two asterisks represent p<0.01; N=3 per condition) FIG. 3 A: CellTiter-Glo cell viability assay (CTG) showed significant decrease in the luminescence signal for all tested lung/LUAD cell lines transfected with siPOOLs against CASP8AP2 (siC8AP2) containing 30 individual sgRNAs to minimize potential off-target effects compared to respective cell lines transfected with negative control siPOOLs (siNC) at 72 hours post-transfection (Student's t-test; three asterisks represent p<0.001; two asterisks represent p<0.01; one asterisk represents p<0.05; N=3-4 per condition)

FIG. 3 B: RT-qPCR confirmed significant siPOOL-mediated CASP8AP2 knockdown in all tested lung and LUAD cell lines transfected with siC8AP2 compared to respective cell lines transfected with siNC at 48 hours post-transfection (Student's t-test; three asterisks represent p<0.001; two asterisks represent p<0.01; N=3 per condition)

FIG. 4: Caspase-Glo luminescent apoptosis assays demonstrated significant activation of effector Caspase-3/7 as well as both extrinsic (Caspase-8) and intrinsic (Caspase-9) caspase cascades in LUAD cell lines NCI-H1975 (H1975) and NCI-H838 (H838), but not in the normal lung cell line IMR-90 transfected with siPOOLs against CASP8AP2 (siC8AP2) compared to respective cell lines transfected with negative control siPOOLs (siNC) at 48 hours post-transfection (Student's t-test; two asterisks represent p<0.01; one asterisk represents p<0.05; N=3 per condition)

FIG. 5, Top panel represents PI staining and BrdU incorporation profiles of cells transfected with negative control siPOOLs (siNC); FIG. 5, middle panel represents cells transfected with siPOOLs against CASP8AP2 (siC8AP2); FIG. 5, bottom panel represents quantification of annotated cell cycle phases and statistical analysis of their deregulation between siC8AP2- and siNC-transfected cells (Student's t-test; three asterisks represent p<0.001; two asterisks represent p<0.01; one asterisk represents p<0.05; N=3.

FIG. 6 A: RT-qPCR results for CDKN1A and HIST2H2BE mRNA levels at 48 hours post-transfection with siPOOLs against CASP8AP2 (siC8AP2) or negative control siPOOLs (siNC). All tested cell lines demonstrated a significant increase in CDKN1A (p21) mRNA levels and HIST2H2BE mRNA (Student's t-test; three asterisks represent p<0.001; two asterisks represent p<0.01; one asterisk represents p<0.05: N=3 per condition)

FIG. 6 B: RT-qPCR results for HIST1H3A, HIST1H2BK and HIST1H3D. All tested cell lines demonstrated a significant decrease in HIST1H3A mRNA levels and no significant change in HIST1H2BK mRNA levels upon siC8AP2 48 hours post-transfection measured by RT-qPCR. Normal lung cell line IMR-90 and LUAD cell line NCI-H1975 demonstrated a significant decrease in HIST1H3D mRNA levels, while no significant change was observed in LUAD cell line NCI-H838.

FIG. 7 A: CellTiter-Glo cell viability assay (CTG) was performed on six additional non-lung cancer cell lines: HCT 116 (colon cancer), MDA-MB-231 (breast cancer), MCF-7 (breast cancer), HepG2 (liver cancer), Panc-1 (pancreatic cancer) and MKN-45 (stomach cancer) at 48 hours post-transfection with siPOOLs against CASP8AP2 (siC8AP2) or negative control siPOOLs (siNC). HCT116 cells showed a strong decrease in cell viability comparable to the average decrease observed in LUAD cells. MDA-MB-231 cells showed a less pronounced effect comparable to the one observed in the Calu-6 LUAD cell line. Three other cell lines also showed a significant decrease in viability. Only MKN-45 cells showed no significant effect (Student's t-test; three asterisks represent p<0.001; two asterisks represent p<0.01; one asterisk represents p<0.05; N=3-4 per condition). The experimental conditions for non-LUAD cancer cell lines recapitulated respective conditions of LUAD experiments described above.

FIG. 7 B: RT-qPCR confirmed significant siPOOLs-mediated CASP8AP2 knockdown in all tested cell lines transfected with siC8AP2 compared to respective cell lines transfected with siNC at 48 hours post-transfection (Student's t-test; three asterisks represent p<0.001; two asterisks represent p<0.01; one asterisk represents p<0.05; N=3 per condition)

FIG. 8: RNAi-based knockdown revealed differential responses to CASP8AP2 silencing between non-transformed lung and NSCLC cell lines.

(A) RT-qPCR to confirm siPOOL-mediated CASP8AP2 knockdown (siC8AP2, 10 nM) in all tested cell lines transfected with siC8AP2 compared to respective cell lines transfected with non-targeting negative control siPOOL (siNC, 10 nM) at 48 hours post-transfection (n=3-6).
(B) CellTiter-Glo cell viability assay (CTG) to assess the effect of CASP8AP2 siPOOL-mediated knockdown (siC8AP2, 10 nM) on non-transformed lung fibroblast cell lines IMR-90 and WI-38, NSCLC LUAD cell lines Calu-6, PC-9, NCI-H1975 (H1975), NCI-H838 (H838), and NSCLC LCLC cell lines NCI-H460 (H460) and NCI-H1299 (H1299) compared to respective cell lines transfected with non-targeting negative control siPOOL (siNC, 10 nM) at 72 hours post-transfection (n=3-4).

For each biological replicate, data were normalized to the respective negative control signal and plotted as individual values. Bar heights represent mean, error bars represent Standard deviation (SD). Significance testing was performed using two-tailed unpaired Student's t-test with Welch's correction., represented as ***, p<0.001; **, p<0.01; *, p<0.05. Significance testing between normal and NSCLC cell lines sensitive to CASP8AP2 silencing was performed using two-tailed unpaired Student's t-test with Welch's correction.

For each biological replicate, data were normalized to the respective negative control signal and plotted as individual values. Bar heights represent mean, error bars represent range. Significance testing was performed on log 2-transformed fold change data using two-tailed paired Student's t-test, represented as ***, p<0.001; **, p<0.01; *, p<0.05. Significance testing between normal and NSCLC cell lines sensitive to CASP8AP2 silencing was performed using two-tailed unpaired Student's t-test with Welch's correction.

### DETAILED DESCRIPTION OF THE INVENTION

Where the term “comprise” or “comprising” is used in the present description and claims, it does not exclude other elements or steps. For the purpose of the present invention, the term “consisting of” is considered to be an optional embodiment of the term “comprising”. If hereinafter a group is defined to comprise at least a certain number of embodiments, this is also to be understood to disclose a group which optionally consists only of these embodiments.

Where an indefinite or a definite article is used when referring to a singular noun e.g. “a” or “an”, “the”, this includes a plural form of that noun unless specifically stated. Vice versa, when the plural form of a noun is used it refers also to the singular form.

Furthermore, the terms first, second, third or (a), (b), (c) and the like in the description and in the claims are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

In the context of the present invention any numerical value indicated is typically associated with an interval of accuracy that the person skilled in the art will understand to still ensure the technical effect of the feature in question. As used herein, the deviation from the indicated numerical value is in the range of ±10%, and preferably of ±5%. The aforementioned deviation from the indicated numerical interval of ±10%, and preferably of ±5% is also indicated by the terms “about” and “approximately” used herein with respect to a numerical value.

According to the first aspect, the present invention provides the use of a Caspase-8 associated protein-2 (CASP8AP2) antagonist for use in the prevention or treatment of cancer, with the proviso that the CASP8AP2 antagonist is not a peptide comprising the amino acid sequence SMMPDELLTSL (SEQ ID NO: 140) or a peptide comprising the amino acid sequence KLDKNPNQV (SEQ ID NO:141).

“CASP8AP2” is the abbreviation for Caspase 8-associated protein. Synonyms for CASP8AP2 are CED-4, FLASH and RIP25. Preferably, CASP8AP2 is of mammalian origin and particularly preferred of human origin. Human CASP8AP2 has the reference sequence Genbank NM_012115.4 providing the mRNA sequence as well as the deduced protein sequence.

“a CASP8AP2 antagonist” herein includes any compound capable of binding to CASP8AP2, in particular human CASP8AP2. The term includes also compounds capable of reducing the transcription and/or translation of the CASP8AP2 gene and/or the stability of the CASP8AP2 mRNA or protein or preventing its interaction with other molecules. Preferably, the CASP8AP2 antagonist is not a peptide consisting of the amino acid sequence SMMPDELLTSL (SEQ ID NO: 140) or a peptide consisting of the amino acid sequence KLDKNPNQV (SEQ ID NO:141).

The binding property of the compound capable of binding to CASP8AP2 may be determined by an ELISA or luminescent assay using CASP8AP2 as bound antigen. Preferably, the CASP8AP2 antagonist binds specifically to CASP8AP2.

In a preferred embodiment the CASP8AP2 antagonist reduces the viability of cancer cells. The “viability of cancer cells” may be determined in vitro or in vivo by methods known in the art. Exemplary in vitro methods include trypan blue exclusion or a luminescence-bases assay such as the CellTiter-Glo assay from the company Promega. Details are described in the Examples section.

In a further preferred embodiment the CASP8AP2 antagonist does not significantly reduce the viability of normal cells in the subject receiving the treatment. The normal cells in the subject receiving the treatment are outside the cancer to be treated, if the cancer is a solid tumor. The viability of a normal cell is “not significantly reduced” if the viability of the treated normal cells is not less than 60% of the viability of the untreated cell. Preferably, not less than 80%, even more preferred not less than 90% of the viability of the untreated cell.

In a particularly preferred embodiment the CASP8AP2 antagonist is an anti-CASP8AP2 antibody specifically binding to said antigen, or a functional fragment thereof. Methods for generating specific antibodies in laboratory animals including mouse, rabbit and goat are well-known as outlined below. Anti-CASP8AP2 antibodies are also commercially available such as a polyclonal rabbit anti-Human FLASH (commercially available from the company Abcam).

Preferably, the antibody neutralizes the binding of CASP8AP2 protein to caspase-3, caspase-7 and/or caspase-8, preferably caspase-8. Such antibodies can be obtained by generating anti-CASP8AP2 antibodies and subsequent screening for neutralizing antibodies. The design of such screening assays is routine experimentation for the person skilled in the art. For example, neutralizing antibodies can be screened using a competitive binding assay format.

The antibody may be polyclonal or monoclonal. The antibody may be an IgG, IgM, IgD, IgA or IgE antibody, with IgG being preferred. Functional binding fragments of the antibody include Fab, F(ab)2 or scFv fragments. The antibodies or functional fragments may be conjugated to produce derivatives. Derivatives may include glycosylation variants or obtained by cross-linking to produce aggregates.

Antibodies may be generated by means known in the art. For example, antibodies can be generated by immunizing laboratory animals. The B cells producing the relevant antibodies can be fused with myeloma cells to produce hybridoma cells which can be taken into culture for the production of the antibodies. Methods for purifying the antibodies from the medium are known. For example, Protein A, Protein G or Protein A/G, Ion exchange Chromatography (IEX) or Hydrophobic interaction chromatography (HIC) are known to the person skilled in the art.

In a further preferred embodiment, the CASP8AP2 antagonist is a caspase antagonist, wherein the caspase is caspase-3, caspase-7, caspase-8 or caspase-9. Preferably, the caspase antagonist is an analog of caspase-3, caspase-7, caspase-8 or caspase-9 capable of blocking the binding of CASP8AP2 with the endogenous caspase-3 (accession No: Genbank AY219866.1), caspase-7 (Genbank BC015799.1), caspase-8 (Genbank AB038985.2) or caspase 9 (Genbank AB019205.2). More preferably, the CASP8AP2 antagonist is a fragment of caspase-3, caspase-7, caspase-8 or caspase-9.

In a further preferred embodiment, the CASP8AP2 antagonist is a low molecular weight compound. Preferably, the compound is capable of reducing the interaction of CASP8AP2 protein with a caspase at the protein level, preferably the caspase is caspase-3, caspase-7 or caspase-8. Most preferred, the caspase is caspase-8. The low molecular weight compound may be obtained by selecting from available chemical libraries. Selection procedures are described in more detail below.

Alternatively, in a further preferred embodiment, a CASP8AP2 antagonist inhibits the transcription and/or translation of the CASP8AP2 gene and/or the stability of the CASP8AP2 mRNA. A CASP8AP2 antagonist is considered to represent an antagonist if it reduces the transcription and/or translation of the CASP8AP2 gene by at least 50%, preferably at least 80%, more preferably at least 90%, even more preferred by at least 95% compared to the situation in the absence of the antagonist.

Means for suppressing the transcription and/or translation of a given gene and methods for producing them are known to the person skilled in the art. The CASP8AP2 antagonist may act by sequence-specific gene silencing.

In a preferred embodiment, the antagonist may be an antisense oligonucleotide. Antisense oligonucleotides act by hybridizing to target mRNA. Depending on backbone modifications of the oligonucleotide, degradation may occur due to RNase H. The design of suitable antisense oligonucleotides for given target sequences is known to the person skilled in the art; see e.g. Aarstma-Rus et al., Molecular Therapy 17 (2009), 548-553. Preferably, the antisense oligonucleotide is capable of binding to and/or is at least partially complementary to a region of the CASP8AP2 gene or regulatory elements in its close vicinity, preferably the CASP8AP2 gene is human.

In a further preferred embodiment, the antagonist may be a ribozyme. Ribozymes are RNA molecules that have intrinsic catalytic activity. The most extensively studied ribozyme is the hammerhead ribozyme. The catalytic motif within the ribozyme is flanked by sequences that are complementary to sequences surrounding the target RNA cleavage site and serve as guides of ribozymes to its mRNA targets.

In a further preferred embodiment, the CASP8AP2 antagonist is a small interfering RNA (siRNA). siRNAs lead to a transient sequence-specific gene silencing. Software for the design of suitable sequences is available to the skilled person; see e.g. Naito et al., Front. Genet. 3 (2012), 1-7. Preferably, the siRNA is capable of interfering with the gene expression of the CASP8AP2 gene and comprises a first strand of RNA at least partially complementary to 15 nucleotides of the CASP8AP2 gene, and a second strand of RNA of 15 to 30 nucleotides in length, wherein at least 12 nucleotides of the first strand and second strands are complementary to each other and form an siRNA duplex.

Preferably, the siRNA is provided in the form of siPOOLs which are complex mixtures of target specific siRNA. More preferably, thirty siRNA (sense and antisense each) are used. Particularly, the siRNA have a nucleic acid sequence selected from any one of the SEQ ID Nos: 20 to 139.

In a further preferred embodiment, the CASP8AP2 antagonist is a short hairpin RNA (shRNA). While an siRNA effect is of transient nature, shRNA allow for high potency and sustainable effects. Design tools for shRNA are also available to the person skilled in the art. After delivery of the shRNA expression vector into the cytoplasm, the vector needs to be transported into the nucleus for transcription; see Rao et al., 61 (2009), 746-759 for a review.

In a further preferred embodiment, the CASP8AP2 antagonist is a microRNA (miRNA). A microRNA is a small non-coding RNA molecule that functions in RNA silencing and post-transcriptional regulation of gene expression. miRNA resemble the siRNAs of the RNAi pathway. Design tools for miRNAs are available to the person skilled in the art; see Chen et al., Briefings in Bioinformatics 20 (2019), 1836-1852 for a review.

In a further preferred embodiment, the CASP8AP2 antagonist is a CRISPR-guide RNA (sgRNA). CRISPR-based genome editing requires two components: a guide RNA and a CRISPR-associated endonuclease protein (Cas) or a derivative or fusion thereof. The guide RNA directs the Cas nuclease to the specific target DNA sequence, which then cuts the DNA at that site resulting in a double-strand break. The cell tries to repair it e.g. via non-homologous end to end joining, which is prone to errors by the insertion or deletion of bases which can lead to protein disruption and is the preferred pathway for knocking out a particular gene. In a further preferred embodiment, the gene expression is inhibited by CRISPR interference (CRISPRi). CRISPRi uses the sequence-specific binding of a Cas9/sgRNA complex to the gene. Since instead of the active Cas9, a variant thereof designated dCas9 is used, which carries specific mutations to inactivate the endonuclease function, the dCas9/sgRNA complex does not cleave DNA strands. Due to the binding of the complex to the DNA strand, gene transcription is inhibited by blocking of RNA polymerases. In a particularly preferred embodiment the dCas9 may further comprise a protein domain of e.g. Kruppel associated box (KRAB), whereby the transcription of the bound gene in human cells is reduced up to 50%, preferably up to 80%, more preferably up to 90%, particularly preferred up to 99%. Design tools for the guideRNA are available to the person skilled in the art; see e.g. from the website of the Broad Institute (https.//portals.broadinstitute.org/gpp/public/analysis-tools/sgrna-design).

Preferably, the sgRNA comprises any of the nucleic acid sequences set forth in SEQ ID Nos: 3 to 5. More preferably, the sgRNA consists of any of the nucleic acid sequences set forth in SEQ ID Nos 3 to 5.

“Cancer” refers to the physiological condition in mammals that is typically characterized by unregulated cell growth. The term herein includes any type of cancer. These types include carcinoma derived from epithelial cells; sarcoma; lymphoma and leukemia; germ cell tumor and blastomas. More particular, from the group consisting of chronic myeloid leukemia (CML), acute lymphoblastic leukemia (ALL), Philadelphia chromosome positive acute lymphoblastic leukemia (Ph+ALL), squamous cell carcinoma, small cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, head and neck cancer, gastric cancer, germ cell tumor, pediatric sarcoma, sinonasal cancer, multiple myeloma, acute myelogenous leukemia (AML), and chronic lymphocytic leukemia (CLL).

Preferably, the cancer is a carcinoma. More preferably the carcinoma is selected from breast, prostate, lung, pancreas and colon. Even more preferably, the cancer is a lung cancer, breast cancer, or pancreas cancer. Particularly preferred, the cancer is a lung adenocarcinoma, squamous cell lung cancer, Estrogen-Receptor (ER)-/Progesteron-Receptor (PR)-positive breast cancer, triple-negative breast cancer, pancreatic ductal adenocarcinoma or hepatocellular carcinoma. Most preferred, the cancer is a lung adenocarcinoma.

In an alternative preferred embodiment, the cancer is not a colon cancer and/or a rectal cancer. In a further alternative preferred embodiment, the cancer is not a stomach cancer (gastric cancer). More preferably, the cancer is neither colon cancer nor rectal cancer.

In a further preferred embodiment, the cancer is a sarcoma. Preferably, the sarcoma is a bone, cartilage, fat or nerve tumor.

In a further preferred embodiment, the cancer is a lymphoma and leukemia.

In a further preferred embodiment, the cancer is germ cell tumor including seminoma and dysgerminoma.

In a further preferred embodiment, the cancer is a blastoma.

In a further preferred embodiment the JunB pathway is not activated in the cancer upon administration of the CASP8AP2 antagonist. The activation of the JunB pathway may be determined by comparing the JUNB mRNA level in the presence of the CASP8AP2 antagonist in the cancer with the JUNB mRNA level in the non-transformed cell. JunB is a component of the AP-1 transcription factor.

The cancer may be in different stages. Currently, it is contemplated that the treatment of the invention is useful at any stage of the cancer.

According to another aspect, the present invention provides a method for preventing or treating cancer comprising administering a CASP8AP2 antagonist to a patient in need thereof. The CASP8AP2 antagonist and the cancer are as defined above. The patient is a mammalian, preferably a human patient.

“preventing or treating” includes any type of a beneficial effect, e.g. amelioration of at least one symptom of a disease or disorder. A beneficial effect can take the form of an improvement over baseline. A beneficial effect can also take the form of arresting, slowing, retarding, or stabilizing of a deleterious progression of a marker of cancer. An effective treatment may e.g. reduce patient pain, reduce the size and/or number of lesions, may reduce or prevent metastasis of a tumor, and/or may slow tumor growth.

The administration may be by any route including intravenous, subcutaneous and oral. Oral is the preferred route of administration. Suitable pharmaceutical compositions are outlined below.

The frequency of administration and dose to be administered is determined by the physician.

If the CASP8AP2 antagonist is a small molecule or a protein, the daily dosage may be in the range from 1 μg to 100 mg, preferably in the range from 10 μg to 10 mg. If the CASP8AP2 antagonist is a nucleic acid, the daily dosage may be in the range from 1 μg to 100 mg, preferably in the range from 10 μg to 10 mg.

The pharmaceutical composition may be administered once, twice or three times daily. The treatment is preferably over a period of at least two weeks, preferably at least four weeks, even more preferred at least 52 weeks.

The above described CASP8AP2 antagonist can be applied as a monotherapy or in combination with known anti-cancer agents. Preferred known anti-cancer agents can be a cytotoxin, a drug or a radioisotope. A cytotoxin or cytotoxic agent includes any agent that is detrimental to (e.g. kills) cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g. methotrexate, 6-mercaptopurine, 6-thioguanine, cytarabine, 5-fluorouracil, decarbazine), alkylating agents (e.g. mechlorethamine, thiotepa, chlorambucil, melphalan, carmustine (BSNU) and lomustine (CCNU), cyclophosphamide, busulfan, dibromomannitol, streptozotocin, mitomycin C, or cis-dichlorodiamine platinum (II) (DDP), cisplatin), anthracyclines (e.g. daunorubicin (formerly daunomycin) or doxorubicin), antibiotics (e.g. dactinomycin (formerly actinomycin), bleomycin, mithramycin, or anthramycin (AMC)), or anti-mitotic agents (e.g., vincristine or vinblastine).

According to another aspect of the present invention, a pharmaceutical composition comprising a pharmaceutically acceptable excipient is provided.

Pharmaceutically acceptable excipients are known to the person skilled in the art. It is known to use antiadherents, binders, coatings, colors, disintegrants, flavors, glidants, lubricants, preservatives, sorbents, sweeteners, and/or vehicles in order to provide suitable dosage forms. Suitable pharmaceutical dosage forms include tablets, capsules, solutions, freeze-dried forms, suppositories, dragees.

According to another aspect, the present invention provides a method of identifying a CASP8AP2 antagonist comprising
i) screening a test compound for binding to CASP8AP2 protein;
ii) optionally determining in vitro the caspase apoptotic activity induced by the test compound identified in step i) in a cancer cell line, wherein the caspase is selected from caspase-3/7, caspase-8 and caspase-9; and
iii) selecting the test compound as a CASP8AP2 antagonist if the test compound binds to CASP8AP2 and optionally increases the apoptotic activity of Caspase-3/7 and/or Caspase-8 and/or Caspase-9 compared to the caspase apoptotic activity in the absence of the test compound.

The test compound may be a small molecule. Preferably, the small molecule is within a complex chemical library. The screening of a test compound as defined in step i) for binding to CASP8AP2 has been outlined above. The screening may include high-throughput techniques known to the person skilled in the art. Step ii) for determining the apoptotic activity of can be carried out using commercially available test kits such as Caspase-Glo 3/7 from the company Promega. Details of the assay protocol are outlined in the Examples section below.

A suitable reference cell line may be a lung adenocarcinoma cell line, preferably NCI-H1975 (ATCC CRL-5908).

An alternative for step ii) is that the apoptotic activity of caspase-8 is determined instead of caspase 3/7 using the commercially available test kit such as Caspase-Glo 8 from the company Promega.

A further alternative for step ii) is that the apoptotic activity of caspase-9 is determined instead of caspase-8 using the commercially available test kit such as Caspase-Glo 9 from the company Promega. Preferably, the caspase apoptotic activity is the apoptotic activity of caspase-8.

According to another aspect, a method is provided for identifying a CASP8AP2 antagonist comprising
i) screening a test compound for binding to CASP8AP2 protein; and
ii) optionally determining in vitro the cell viability of a cancer cell in the presence of the test compound; and
iii) selecting the test compound as a CASP8AP2 antagonist if the test compound binds to CASP8AP2 and optionally reduces the cell viability of the cancer cell compared to the cell viability in the absence of the test compound.

Step i) is performed as outlined above. Step ii) may be performed using the classical trypan blue exclusion method. Alternatively, the CellTiter-Glo cell viability assay from the company Promega following the manufacturer's instructions may be used.

Preferably, the cancer cell is the lung adenocarcinoma cell line NCI-H1975 (ATCC CRL-5908).

According to another aspect, a method for preparing a pharmaceutical composition is provided comprising identifying a CASP8AP2 antagonist by the methods of the invention and mixing the CASP8AP2 antagonist with a pharmaceutically acceptable excipient. The methods and pharmaceutically excipients are as described above.

Further preferred embodiments of the present invention relate to:
1. A Caspase-8 associated protein-2 (CASP8AP2) antagonist for use in the prevention or treatment of cancer.
2. The CASP8AP2 antagonist for use according to embodiment 1, wherein the CASP8AP2 antagonist reduces the viability of the cancer cells.
3. The CASP8AP2 antagonist for use according to embodiment 1 or 2, wherein the CASP8AP2 antagonist does not significantly reduce the viability of normal cells in the subject receiving the treatment.
4. The CASP8AP2 antagonist for use according to any one of embodiments 1 to 3, with the proviso that said cancer is not stomach cancer and/or colon cancer.
5. The CASP8AP2 antagonist for use according to any one of embodiments 1 to 4, wherein the cancer is selected from the group consisting of lung cancer, breast cancer, pancreatic cancer or liver cancer.
6. The CASP8AP2 for use according to any one of embodiments 1 to 5, wherein the cancer is a lung adenocarcinoma, squamous cell lung cancer, ER-/PR-positive breast cancer, triple-negative breast cancer, pancreatic ductal adenocarcinoma or hepatocellular carcinoma, preferably, the cancer is a lung adenocarcinoma.
7. The CASP8AP2 antagonist for use according to any of embodiments 1 to 6, wherein the CASP8AP2 antagonist reduces the transcription and/or translation of the CASP8AP2 gene and/or the stability of the CASP8AP2 mRNA or protein.
8. The CASP8AP2 antagonist for use according to embodiment 7, wherein the antagonist is selected from an antisense oligonucleotide, a small interfering RNA (siRNA), a short hairpin RNA (shRNA), a microRNA (miRNA) or a CRISPR-guide RNA (sgRNA).
9. The CASP8AP2 antagonist for use according to embodiment 8, wherein the antisense oligonucleotide is capable of binding to and/or is at least partially complementary to a region of the CASP8AP2 gene or regulatory elements in its close vicinity, preferably the CASP8AP2 gene is human.
10. The CASP8AP2 antagonist for use according to embodiment 8 wherein the siRNA is capable of interfering with the gene expression of the CASP8AP2 gene and comprises a first strand of RNA at least partially complementary to 15 nucleotides of the CASP8AP2 gene, and a second strand of RNA of 15 to 30 nucleotides in length, wherein at least 12 nucleotides of the first strand and second strands are complementary to each other and form a siRNA duplex.
11. The CASP8AP2 antagonist for use according to embodiment 8, wherein the sgRNA is at least partially complementary to 15 nucleotides of the CASP8AP2 gene and in addition a CRISPR protein lacking endonuclease activity is administered, preferably the CRISPR protein is fused to at least a domain of Kruppel associated box (KRAB) protein.
12. The CASP8AP2 antagonist for use according to any one of embodiments 1 to 6, wherein the CASP8AP2 antagonist is an antibody specifically binding to CASP8AP2 protein, or a binding fragment thereof.
13. The CASP8AP2 antagonist for use according to embodiment 12, wherein the antibody specifically binding to CASP8AP2 at least partially blocks the binding of CASP8AP2 to a caspase selected from caspase-3, caspase-7, caspase-8 or caspase-9, preferably the caspase is caspase-8.
14. A method for preventing or treating cancer comprising administering a CASP8AP2 antagonist as defined in any one of embodiments 1 to 13 to a patient in need thereof, preferably the patient is human.
15. The method according to embodiment 14, wherein the CASP8AP2 antagonist reduces the viability of the cancer cells.
16. The method according to embodiment 14 or 15, wherein the CASP8AP2 antagonist does not significantly reduce the viability of the normal cells in the subject receiving the treatment.
17. The method according to any one of embodiments 14 to 16, with the proviso that said cancer is not stomach cancer and/or colon cancer.
18. The method according to any one of embodiments 14 to 17, wherein the cancer is selected from the group consisting of lung cancer, breast cancer, pancreatic cancer or liver cancer.
19. The method according to any one of embodiments 14 to 18, wherein the cancer is a lung adenocarcinoma, squamous cell lung cancer, ER-/PR-positive breast cancer, triple-negative breast cancer, pancreatic ductal adenocarcinoma or hepatocellular carcinoma, preferably the cancer is a lung adenocarcinoma.
20. A pharmaceutical composition comprising a CASP8AP2 antagonist as defined in any one of embodiments 1 to 13 and a pharmaceutically acceptable excipient.
21. A method for identifying a CASP8AP2 antagonist comprising
i) screening a test compound for binding to CASP8AP2 protein;
ii) optionally determining in vitro the caspase-mediated apoptotic activity induced by the test compound identified in step i) in a cancer cell line, wherein the caspase is selected from caspase-3/7, caspase-8 and caspase-9; and
iii) selecting the test compound as a CASP8AP2 antagonist if the test compound binds to CASP8AP2 and optionally increases the apoptotic activity of Caspase-3/7 and/or Caspase-8 and/or Caspase-9 compared to the caspase-mediated apoptotic activity in the absence of the test compound.
22. The method according to embodiment 21, wherein the selected test compound increases the caspase-mediated apoptotic activity by at least 10%, preferably at least 20%, more preferably at least 50% compared to the caspase-mediated apoptotic activity in the absence of the test compound.
23. The method according to embodiment 21 or 22, wherein the cancer cell is selected from the lung adenocarcinoma cell line NCI-H1975 (ATCC CRL-5908) and the caspase-mediated apoptotic activity is the apoptotic activity of caspase-8.
24. A method for identifying a CASP8AP2 antagonist comprising
i) screening a test compound for binding to CASP8AP2 protein; and
ii) optionally determining in vitro the cell viability of a cancer cell induced by the test compound identified in step i); and
iii) selecting the test compound as a CASP8AP2 antagonist if the test compound binds to CASP8AP2 and optionally reduces the cell viability of the cancer cell compared to the cell viability of the cancer cell in the absence of the test compound.
25. The method of embodiment 24, wherein the cancer cell is NCI-H1975 (ATCC CRL-5908).
26. A method for preparing a pharmaceutical composition comprising a CASP8AP2 antagonist comprising identifying a CASP8AP2 antagonist by the method of any one of embodiments 21 to 25 and mixing the CASP8AP2 antagonist with at least one pharmaceutically acceptable excipient.

The invention is further described in the following examples which are solely for the purpose of illustrating specific embodiments of the invention, and are also not to be construed as limiting the scope of the invention in any way.

### EXAMPLES

### Example 1: CRISPRi-Dropout Screen Identified CASP8AP2 as an Essential Viability Factor for Lung Adencarcinoma (LUAD) Cell Lines

### Material and Methods

### A) CRISPRi Screen Workflow

42000 sgRNAs were designed and ordered against 4633 transcription start sites (TSSs; also referred to as “transcripts” for simplification) corresponding to 2058 genes differentially expressed between lung adenocarcinoma (LUAD) and normal lung patient samples from TCGA and TANRIC databases as well as 30 positive control genes with annotated functionality and 10 known LUAD driver genes. The resulting sgRNA-encoding oligonucleotide library (Twist Bioscience) was cloned into the LentiCRISPRv2-dCas9-KRAB(iv)-puro vector using Gibson assembly, maintaining high coverage at every cloning step (resulting number of colony forming units (cfu) per sgRNA was >2000). The plasmid library was transfected into HEK293T cells along with the packaging vector psPAX and the envelope vector pMD2.G to produce a high-coverage lentiviral library.

The lentiviral library was transduced into eight LUAD cell lines (Calu-6, PC-9, NCI-H1975, NCI-H838, NCI-H1650, NCI-H522, NCI-H3122 and HCC827) at low multiplicity of infection (MOI ˜0.3) to ensure that every cell was transduced with one sgRNA at max, and high coverage (500 or 1000 cells per sgRNA after antibiotic selection) to ensure statistically powerful representation of all sgRNAs. After transduction, cells were subjected to 48 hours of puromycin selection. The resulting cell pool was maintained in culture for 21 days after transduction at minimum coverage of 500× or 1000×. At 21 days (endpoint), cells were harvested, pelleted and snap-frozen for subsequent genomic DNA (gDNA) isolation. Two independent screen replicates were performed for each cell line.

sgRNA-inserts were PCR-amplified from the gDNA (endpoint samples) and the plasmid library (reference samples) and subjected to next generation sequencing (NGS). sgRNA representation in reference and endpoint samples, identification of depleted sgRNAs and transcripts was performed with the MAGeCK pipeline (Li, W, Xu, H, Xiao, T et al. MAGeCK enables robust identification of essential genes from genome-scale CRISPR Cas9 knockout screens. Genome Biol 15, 554 (2014).

### Results

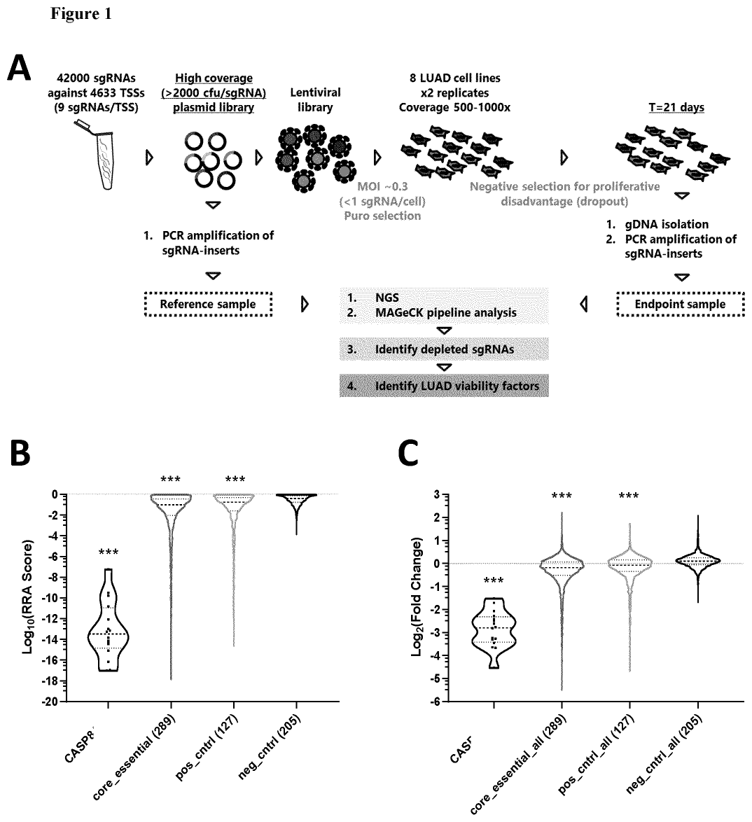

Robust rank aggregation (RRA) scores for negative selection and fold-change in sgRNA-corresponding read counts between all endpoint and reference samples for CASP8AP2, core essential gene transcripts (N=289), positive control gene transcripts (N=127) versus non-targeting sgRNAs aggregated computationally to negative control transcripts (N=205) demonstrated that the sgRNAs targeting CASP8AP2 were significantly depleted from the pool at the screen endpoint, suggesting the essential role of CASP8AP2 in lung cancer viability (Student's t-test; three asterisks represent p<0.001), see FIGS. 1 B and C.

### Example 2: Knockdown of CASP8AP2 with Three Individual sgRNAs Demonstrated Significant Decrease in Cell Viability of LUAD Cell Lines Calu-6, NCI-H1975 (H1975) and NCI-H838 (H838)

### Material and Methods

sgRNA Selection, Cloning and Lentivirus Production

Individual sgRNAs used in the CRISPRi screen were ranked by their performance score calculated by the MAGeCK count pipeline (Li et al., cited above); three sgRNAs for CASP8AP2 (ENST00000552401) with highest cumulative performance score across all replicates of all screened cell lines were selected for validation of the screen results.

The sgRNA sequences were:
sgNC_1 (control):
(SEQ ID NO: 1)
GGGCGAGGAGCTGTTCACCG
sgNC_2 (control):
(SEQ ID NO: 2)
GAGCTGGACGGCGACGTAAA
sgC8AP2_1:
(SEQ ID NO: 3)
GATGCCAGGGAGACCTCGGT
sgC8AP2_2:
(SEQ ID NO: 4)
GCTGCCCGGCCCAAGACAACC
sgC8AP2_3:
(SEQ ID NO: 5)
GCGGTTCCTTTCTGCCCACCG

Individual sgRNA constructs were synthesized in the form of desalted oligonucleotides with Esp3I-compatible sticky ends by Sigma-Aldrich and cloned into LentiCRISPRv2-dCas9-KRAB(iv)-puro vector by restriction digestion of the vector with Esp3I and subsequent ligation with sgRNA-oligonucleotides. Lentivirus was produced by PEI-mediated transfection of the cloned sgRNA-containing constructs, packaging vector psPAX2 and envelope vector pMD2.G into HEK293T cells.

### Lentiviral Transduction of Lung Adenocarcinoma (LUAD) Cell Lines

Calu-6, NCI-H1975 and NCI-H838 cell lines (each obtained from ATCC) were routinely cultured in RPMI-1640 medium supplemented with 10% FCS (Gibco). For transduction, cells were seeded on 24-well plates and 24 hours later transduced with respective lentiviral stocks in presence of 8 μg/mL polybrene. 20 hours later, lentivirus-containing medium was removed, cells were washed with PBS and split in 1:3 ratio on the same plate; remaining two thirds of cell suspensions were seeded on 6-well plates for subsequent RNA isolation. 24 hours later, medium on both 24- and 6-well plates was exchanged to medium supplemented with puromycin (0.5 μg/mL for Calu-6 and NCI-H838, 1 μg/mL for NCI-H1975) for selection of successfully transduced cells. Puromycin was kept in the medium until the end of the experiment and was refreshed every 48 hours.

48 hours after the addition of puromycin (timepoint of complete death of untransduced control cells), Calu-6 and NCI-H838 cells seeded on 6-well plates were lysed using RNA lysis buffer from Quick-RNA Mini/Microprep Kit (Zymo Research) (total T=4 days after transduction). NCI-H1975 cells were lysed for RNA isolation 72 hours after the start of selection (total T=5 days after transduction). RNA from Calu-6 was isolated using Quick-RNA Miniprep Kit, RNA from NCI-H1975 and NCI-H838 was using Quick-RNA Microprep Kit (Zymo Research) according to manufacturer's instructions, with the DNase treatment prolonged to 30 minutes. 24 hours after addition of puromycin to 24-well plates, Calu-6 cells were split in 1:4 ratio back to the same plate in puromycin-supplemented medium. 72 hours later, cells were proportionally seeded in puromycin-supplemented medium on transparent 96-well plates to maintain cell number ratios across different transductions (12% of cells from each well on the 24-well plate were equally divided between three wells of 96-well plate). 48 hours later, CellTiter-Glo assay (Promega) was performed on Calu-6 cells (total T=8 days from transduction).

96 hours after addition of puromycin to 24-well plates, NCI-H1975 cells were split in 1:3 ratio back to the same plate in puromycin-supplemented medium. 72 hours later, transduced NCI-H1975 cells were proportionally seeded in puromycin-supplemented medium on transparent 96-well plates (12% of cells from each well on the 24-well plate were equally divided between three wells of 96-well plate). 96 hours later, CellTiter-Glo assay was performed on NCI-H1975 cells (total T=13 days from transduction).

48 hours after addition of puromycin to 24-well plates, transduced NCI-H838 cells were proportionally seeded on transparent 96-well plates (16% of cells from each well on the 24-well plate were equally divided between three wells of 96-well plate). 72 hours later, CellTiter-Glo assay was performed on NCI-H838 cells (total T=7 days from transduction).

### CellTiter-Glo Cell Viability Assay

CellTiter-Glo cell viability assay (Promega) was performed on 96-well plates according to the manufacturer's protocol using the CellTiter-Glo reagent diluted 1:4 in PBS. Each biological replicate was performed in technical triplicates. For each biological replicate, data were normalized to the respective negative control signal and plotted as individual values. Bar heights represent mean, error bars represent Standard deviation (SD). Significance testing was performed using two-tailed unpaired Student's t-test with Welch's correction., represented as ***, p<0.001; **, p<0.01; *, p<0.05.

### RT-qPCR

RNA was reverse-transcribed using the RevertAid Reverse Transcriptase and random hexamer primers (Thermo Fisher Scientific) according to the manufacturer's instructions. Real-time quantitative PCR (qPCR) was performed using 2× Power SybrGreen Master Mix (Applied Biosystems) and an Applied Biosystems StepOnePlus cycler in technical triplicates. GAPDH was used as a housekeeping gene for the 2^{-ddct} normalization. Primer sequences were:

CASP8AP2-FW:
(SEQ ID NO: 6)
ATCATGGCAGCAGATGATGA
CASP8AP2-RV:
(SEQ ID NO: 7)
CCCAGCGTATATGTCCAGTG
GAPDH-FW:
(SEQ ID NO: 8)
GTGAAGGTCGGAGTCAACG
GAPDH-RV:
(SEQ ID NO: 9)
TGAGGTCAATGAAGGGGTC

### Results

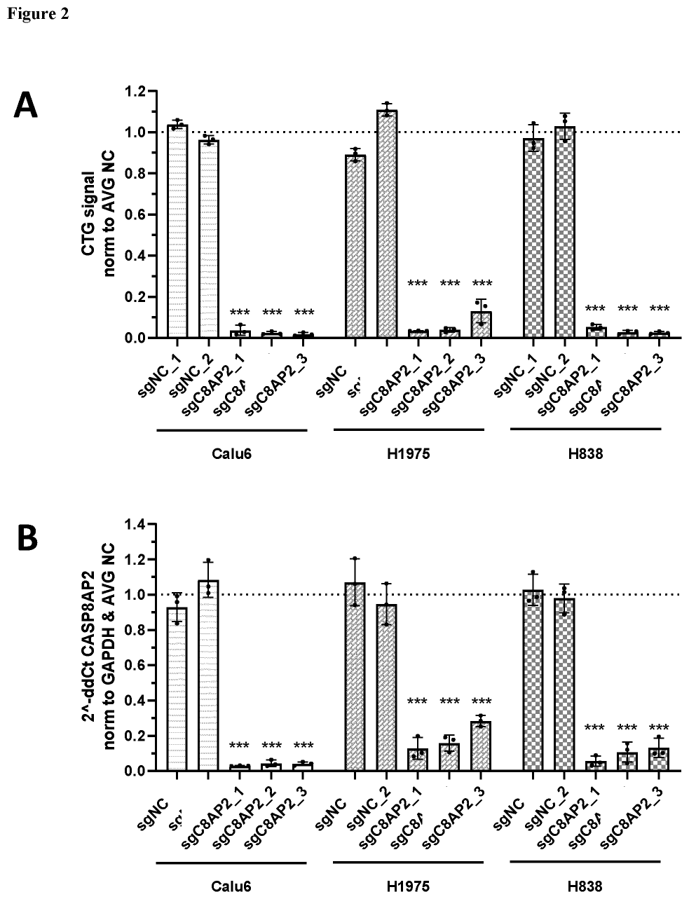

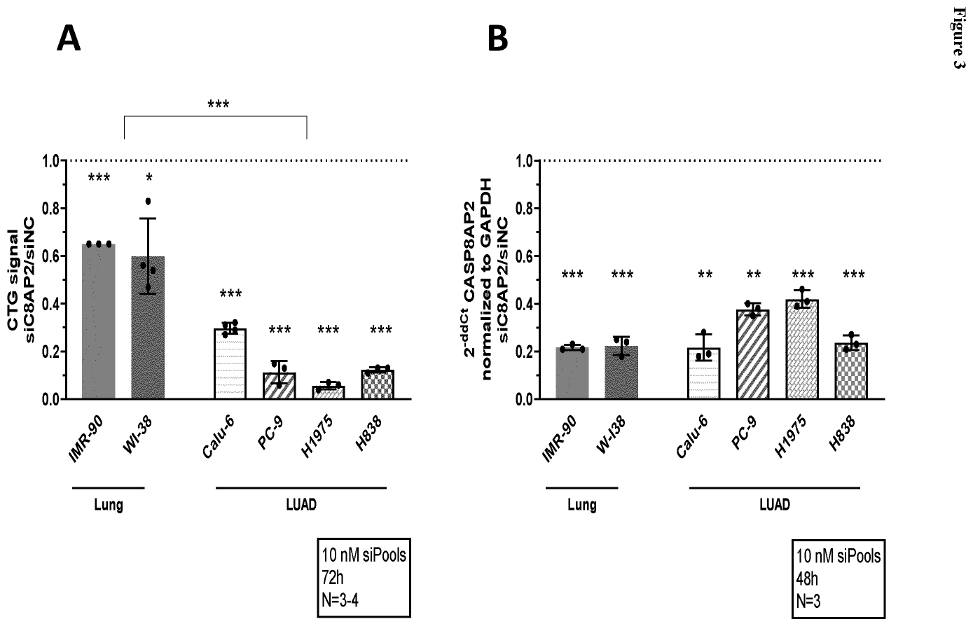

A) CellTiter-Glo cell viability assay (CTG) showed significant decrease in luminescence signal in LUAD cells transduced independently with three constructs expressing sgRNAs against CASP8AP2 (sgC8AP2) compared to two negative control sgRNAs (sgNC) (Student's t-test; three asterisks represent p<0.001; N=3 per condition); see FIG. 2 A.

B) RT-qPCR confirmed significant decrease in CASP8AP2 expression upon knockdown with three independent sgRNAs against CASP8AP2 (sgC8AP2) compared to two negative control sgRNAs (sgNC) (Student's t-test; three asterisks represent p<0.001; two asterisks represent p<0.01; N=3 per condition); see FIG. 2 B.

### Example 3: RNAi-Mediated Knockdown of CASP8AP2 with siPOOLs

### Materials and Methods

siPOOLs Transfection and CellTiter-Glo Cell Viability Assay

Normal (non-transformed) lung cell lines IMR-90 and WI-38 cell lines (obtained from ATCC) were routinely cultured in MEM Eagle medium (Pan Biotech) supplemented with 10% FCS (Gibco). All LUAD cell lines were routinely cultured in RPMI-1640 medium supplemented with 10% FCS (Gibco).

Cells were reverse-transfected with respective siPOOLs (designed and manufactured by siTOOLs Biotech; DNA sequences encoding the sense and antisense strands of the 30 siRNAs of siPool1 are given in SEQ ID NO:20-79; DNA sequences encoding the sense and antisense strands of the 30 siRNAs of siPool2 are given in SEQ ID NO:80-139) at 10 nM final concentration on transparent 96-well plates in technical triplicates, using Lipofectamine RNAiMax (Invitrogen) as a transfection reagent. At 72 hours post-transfection, CellTiter-Glo cell viability assay (Promega) was performed according to the protocol described above.

siPOOLs Transfection for RNA Isolation and RT-qPCR

Cells were reverse-transfected with respective siPOOLs (designed and manufactured by siTOOLs Biotech; DNA sequences encoding the sense and antisense strands of the 30 siRNAs of siPool1 are given in SEQ ID NO:20-79; DNA sequences encoding the sense and antisense strands of the 30 siRNAs of siPool2 are given in SEQ ID NO:80-139) at 10 nM final concentration on 6-well plates, using cell numbers and reagent amounts proportionally scaled-up from the 96-well format. At 48 hours post-transfection, cells were lysed with RNA Lysis buffer from Quick-RNA Microprep Kit (Zymo Research). Subsequently, RNA isolation was performed according to the manufacturer's protocol. RNA was reverse-transcribed and RT-qPCR was performed according to the protocol described above.

### Results

RNAi-mediated knockdown of CASP8AP2 with siPOOLs demonstrated a significant decrease in cell viability of lung adenocarcinoma (LUAD) cell lines Calu-6, PC-9, NCI-H1975 (H1975) and NCI-H838 (H838) compared to the viability of non-transformed lung cell lines IMR-90 and WI-38.

A) CellTiter-Glo cell viability assay (CTG) showed significant decrease in the luminescence signal for all tested lung/LUAD cell lines transfected with siPOOLs against CASP8AP2 (siC8AP2) compared to respective cell lines transfected with negative control siPOOLs (siNC) at 72 hours post-transfection (Student's t-test; three asterisks represent p<0.001; two asterisks represent p<0.01; one asterisk represents p<0.05; N=3-4 per condition); see FIG. 3 A.

B) RT-qPCR confirmed significant siPOOLs-mediated CASP8AP2 knockdown in all tested lung and LUAD cell lines transfected with siC8AP2 compared to respective cell lines transfected with siNC at 48 hours post-transfection (Student's t-test; three asterisks represent p<0.001; two asterisks represent p<0.01; N=3 per condition); see FIG. 3 B.

### Example 4: Caspase-Glo Luminescent Apoptosis Assays

### Materials and Methods

siPOOLs Transfection and Caspase-Glo Apoptosis Assays

Cells were reverse-transfected with the respective siPOOLs (designed and manufactured by siTOOLs Biotech; DNA sequences encoding the sense and antisense strands of the 30 siRNAs of siPool1 are given in SEQ ID NO:20-79; DNA sequences encoding the sense and antisense strands of the 30 siRNAs of siPool2 are given in SEQ ID NO:80-139) at 10 nM final concentration on white 96-well plates in technical duplicates. Two wells per medium type were filled with medium only for obtaining background reads (mixed with same volume of OptiMem used in experimental wells for mixing the transfection reagents).

At 48 hours post-transfection, Caspase-Glo assays (Caspase-Glo 3/7, Caspase-Glo 8 and Caspase-Glo 9, Promega) were performed according to manufacturer's instructions. Briefly, Caspase-Glo reagents were mixed from the provided substrates and buffers (supplied with MG-132 for Caspase-Glo 8 and Caspase-Glo 9 as recommended), aliquoted into opaque tubes and stored at −80° C. The reagents were equilibrated to room temperature for 1.5 hours before the assay. Each aliquot has only been used once to avoid signal loss due to repeated freezing-thawing.

Plates were equilibrated to room temperature for 30 minutes before addition of an equal volume (100 μL/well) of Caspase-Glo reagents. Plates were then placed on an orbital shaker for two minutes at 400 rpm protected from light to facilitate cell lysis, and incubated for one hour at room temperature to stabilize the luminescent signal before readout. Respective background reads were subtracted from experimental reads. Further details of the assay protocol can be found on https://www.promega.de/-/media/files/resources/protocols/technical-bulletins/101/caspase-glo-3-7-assay-protocol.pdf?la=en.

### Results

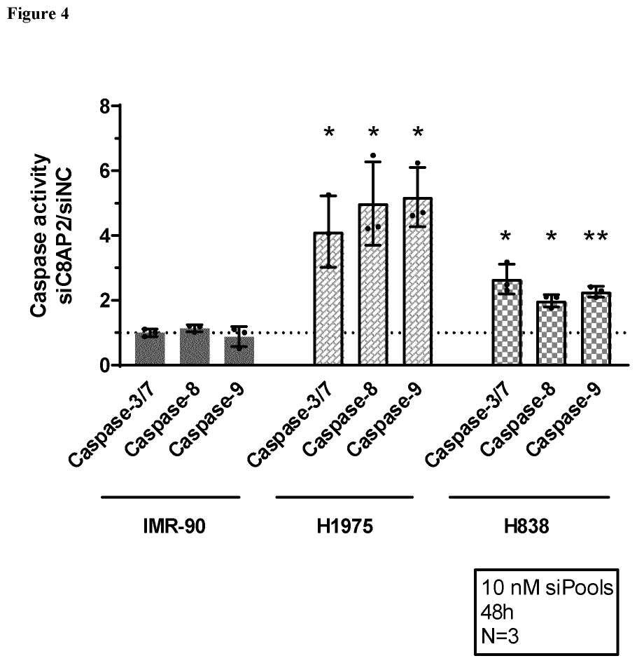

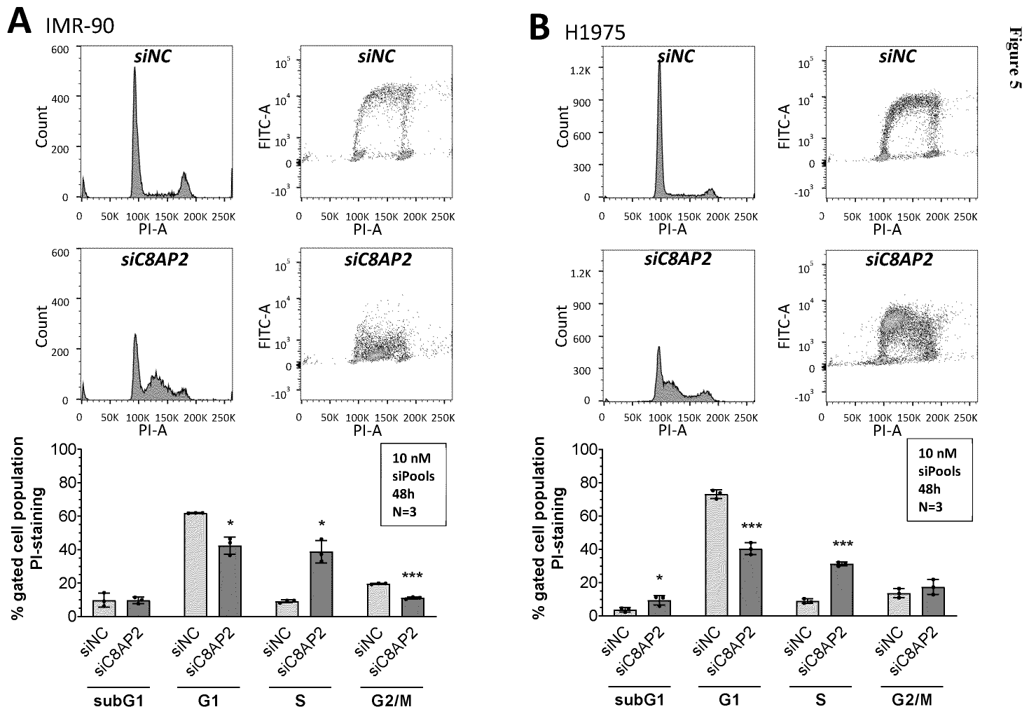

Caspase-Glo luminescent apoptosis assays demonstrated significant activation of effector Caspase-3/7 as well as both extrinsic (Caspase-8) and intrinsic (Caspase-9) caspase cascades in LUAD cell lines NCI-H1975 (H1975) and NCI-H838 (H838), but not in normal lung cell line IMR-90 transfected with siPOOLs against CASP8AP2 (siC8AP2) compared to respective cell lines transfected with negative control siPOOLs (siNC) at 48 hours post-transfection (Student's t-test; two asterisks represent p<0.01; one asterisk represents p<0.05; N=3 per condition); see FIG. 4.

### Example 5: BrdU Incorporation/Propidium Iodide (PI) Staining-Based Cell Cycle Analysis by Flow Cytometry

### Materials and Methods

siPOOLs Transfection and BrdU Incorporation

Cells were reverse-transfected with the respective siPOOLs (designed and manufactured by siTOOLs Biotech; DNA sequences encoding the sense and antisense strands of the 30 siRNAs of siPool1 are given in SEQ ID NO:20-79; DNA sequences encoding the sense and antisense strands of the 30 siRNAs of siPool2 are given in SEQ ID NO:80-139) at 10 nM final concentration on 10 cm cell culture dishes, using cell numbers and reagent amounts proportionally scaled-up from the 96-well format. At 46 hours post-transfection, BrdU was added to the cell culture medium to the final concentration of 10 μM. After two hours (48 hours post-transfection), medium was removed, cells were washed with PBS, trypsinized, collected in 15 mL tubes and pelleted. Cell pellets were washed twice in PBS, resuspended in 1 mL PBS and fixed by addition of 2.5 mL of ice-cold 100% ethanol under vortexing followed by an overnight incubation at 4° C. For storage, cells were transferred to −20° C.

### Anti-BrdU/PI-Staining and Flow Cytometry Analysis

Fixed cells were pelleted, washed in PBS supplemented with 0.5% BSA (w/v) and incubated in 2M HCl for 20 minutes to facilitate DNA hydrolysis, followed by another wash and two minute incubation in 0.1 M sodium tetraborate buffer, pH 8.5. Then, cells were washed again, pelleted and incubated with a FITC-labeled anti-BrdU antibody (#364104, Biolegend) for one hour at room temperature protected from light. Afterwards, samples were washed, pelleted and incubated with 50 ng/mL propidium iodide solution supplemented with 100 μg/mL RNase A for 10 minutes at 37° C. protected from light. Samples were kept on ice, protected from light and analyzed by flow cytometry on a BD Fortessa FACS immediately. For the general cell cycle phase analysis and BrdU incorporation analysis, acquired signals were gated for single cells using FSC-A/SSC-A and FSC-W/FSC-H-based exclusion criteria. For the subG1-analysis, the ungated total cell population was used. The analysis was carried out in the FlowJo software.

### Results

BrdU incorporation/Propidium Iodide (PI) staining-based cell cycle analysis by flow cytometry demonstrated differences between the cell cycle progression in normal lung cell line IMR-90 with a BrdU-negative fraction in S-phase (A) and cell cycle progression in a BrdU-positive fraction in S-phase in LUAD cell lines NCI-H1975 (H1975, B) and NCI-H838 (H838, C) upon siPOOLs-mediated CASP8AP2 knockdown at 48 hours post-transfection.

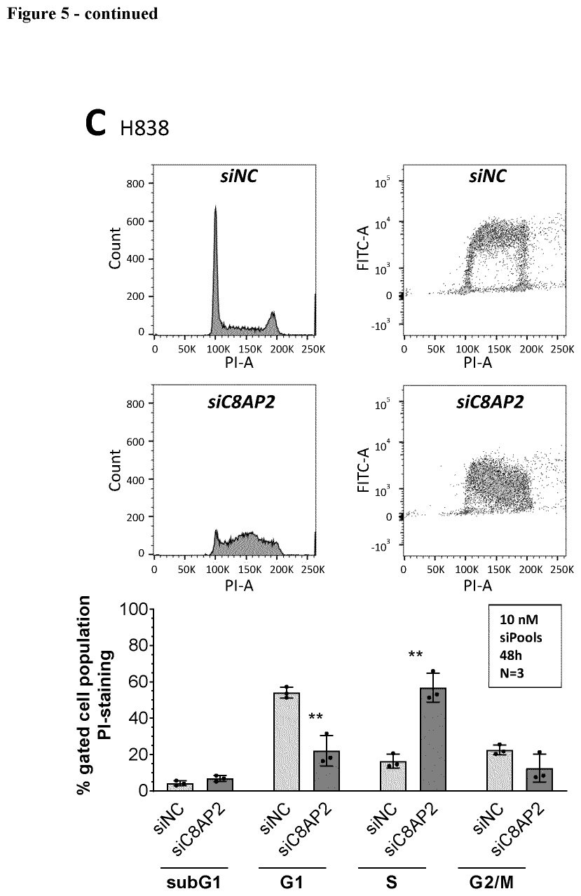

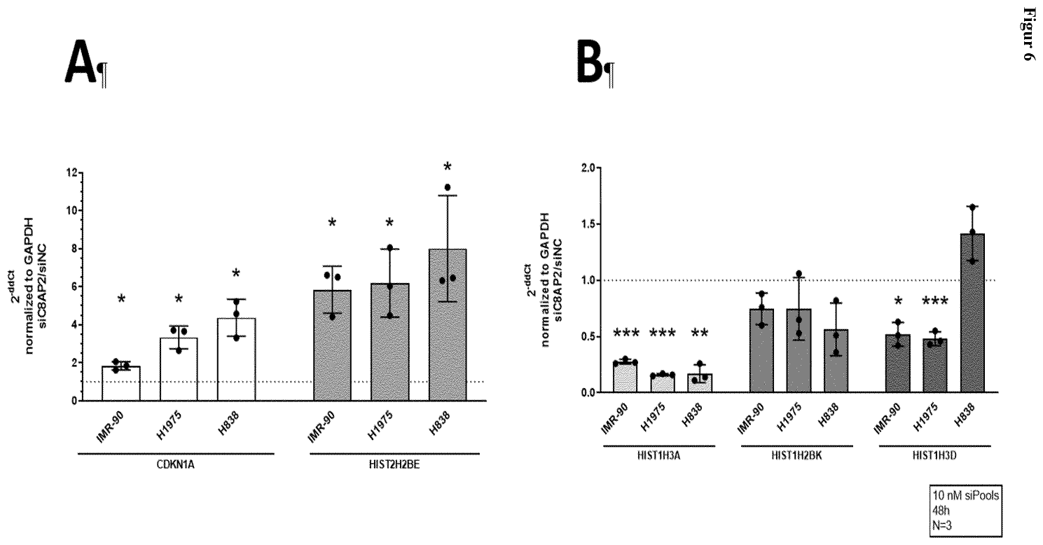

FIG. 5, Top panel represents PI staining and BrdU incorporation profiles of cells transfected with negative control siPOOLs (siNC); FIG. 5, middle panel represents cells transfected with siPOOLs against CASP8AP2 (siC8AP2); FIG. 5, bottom panel represents quantification of annotated cell cycle phases and statistical analysis of their deregulation between siC8AP2- and siNC-transfected cells (Student's t-test; three asterisks represent p<0.001; two asterisks represent p<0.01; one asterisk represents p<0.05; N=3. Significantly increased fraction of NCI-H1975 cells in subG1-phase upon siPOOLs-mediated CASP8AP2 knockdown suggested an increase in apoptosis rates. The same trend was observed in NCI-H838 cells, whereas no difference in subG1 cell percentage was detected in IMR-90 cells.

### Example 6: Normal Lung Cell Line IMR-90 and LUAD Cell Lines NCI-H1975 (H1975) and NCI-H838 (H838) Showed Similar Patterns of Increased p21 mRNA Levels and (De)Regulation of Histone mRNA Levels Upon siPOOLs-Mediated CASP8AP2 Knockdown

### Materials and Methods

siPOOLs Transfection, RNA Isolation and RT-qPCR

The procedure was carried out as described above. RT-qPCR was performed using the following primers:

CDKN1A_483FW:
(SEQ ID NO: 10)
AGGTGGACCTGGAGACTCTCAG
CDKN1A_577RV:
(SEQ ID NO: 11)
TCCTCTTGGAGAAGATCAGCCG
HIST1H2BE-202_704FW:
(SEQ ID NO: 12)
GGCTCTTCGTGTGAGGATTC
HIST1H2BE-202_817FW:
(SEQ ID NO: 13)
CTGGGAGGTAGAGGTTGTAATGA
HIST1H3A-201_252FW:
(SEQ ID NO: 14)
TTTCCAGAGCTCCGCTGT
HIST1H3A-201_322RV:
(SEQ ID NO: 15)
TGTCCTCAAATAGCCCTACCA
HIST1H2BK-201_274FW:
(SEQ ID NO: 16)
TCCAGGGAGATCCAGACG
HIST1H2BK-201_366RV:
(SEQ ID NO: 17)
GTACTTGGTGACGGCCTTG
HIST1H3D-201_67FW:
(SEQ ID NO: 18)
ACCAAGGCTGCTCGAAAG
HIST1H3D-201_127RV:
(SEQ ID NO: 19)
GGTAACGGTGGGGCTTCT

### Western Blot

Cells were transfected with siPOOLs in a 6-well format as described above and were washed at 48 hours post-transfection with cold PBS and lysed in cold RIPA buffer supplemented with phosphatase and protease antagonists. Buffer was added to the cells on ice, distributed across the wells with cell scrapers and incubated on ice for 30 minutes. Lysates were collected into 1.5 mL tubes and centrifuged at top speed in a tabletop centrifuge at 4° C. for 15 minutes to remove debris. Cleared lysates were transferred to new tubes and stored at −80° C.

Protein concentrations were determined using BCA assay. 20 or 30 μg of protein per sample were separated by SDS-PAGE and blotted onto 0.45 μm PVDF membranes (Merck). Primary antibodies against histone H3 (#9715, Cell Signaling Technology) and alpha/beta-tubulin (#2148, Cell Signaling Technology) were used in a 1:1000 dilution; secondary antibody (peroxidase-goat anti-rabbit #111-035-144, Jackson ImmunoResearch) was used in a 1:10000 dilution. Membranes were developed using SuperSignal West Pico Plus chemiluminescent substrate (Thermo Fischer) and the signals were monitored in the ECL ChemoCam Imager (Intas). ImageJ software was used for quantification.

### Results

Normal lung cell line IMR-90 and LUAD cell lines NCI-H1975 (H1975) and NCI-H838 (H838) showed similar patterns of increased p21 mRNA levels and (de)regulation of histone mRNA levels upon siPOOLs-mediated CASP8AP2 knockdown.

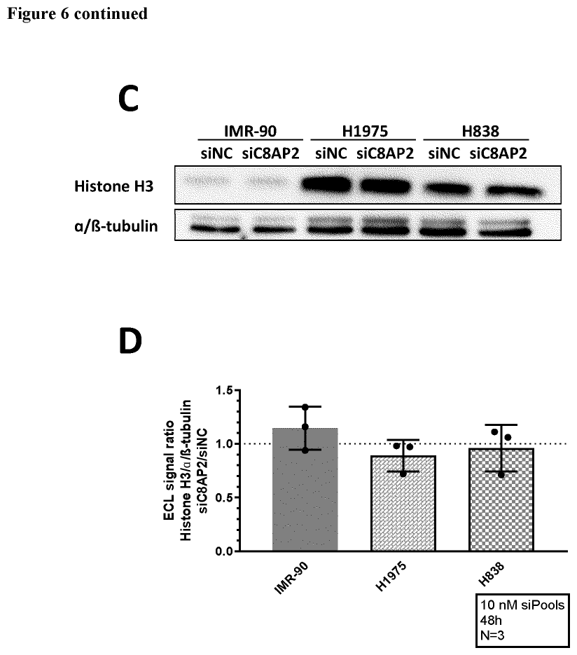

A) RT-qPCR results for CDKN1A and HIST2H2BE mRNA levels at 48 hours post-transfection with siPools against CASP8AP2 (siC8AP2) or negative control siPools (siNC). All tested cell lines demonstrated a significant increase in CDKN1A (p21) mRNA levels and HIST2H2BE mRNA (Student's t-test; three asterisks represent p<0.001; two asterisks represent p<0.01; one asterisk represents p<0.05: N=3 per condition); see FIG. 6 A.

B) RT-qPCR results for HIST1H3A, HIST1H2BK and HIST1H3D. All tested cell lines demonstrated a significant decrease in HIST1H3A mRNA levels and no significant change in HIST1H2BK mRNA levels upon siC8AP2 48 hours post-transfection measured by RT-qPCR. Normal lung cell line IMR-90 and LUAD cell line NCI-H1975 demonstrated a significant decrease in HIST1H3D mRNA levels, while no significant change was observed in LUAD cell line NCI-H838; see FIG. 6 B.

C) Western Blot analysis and D) respective quantification of histone H3 expression in siC8AP2- and siNC-transfected cells. No changes were observed in protein levels of histone H3 in all tested cell lines (Student's t-test; N=3); see FIGS. 6 C and D.

### Example 7: siPOOLs-Mediated Knockdown of CASP8AP2 Demonstrated Non-Uniform Effects on Cell Viability of Non-Lung Cancer Cell Lines

### Materials/Methods and Results

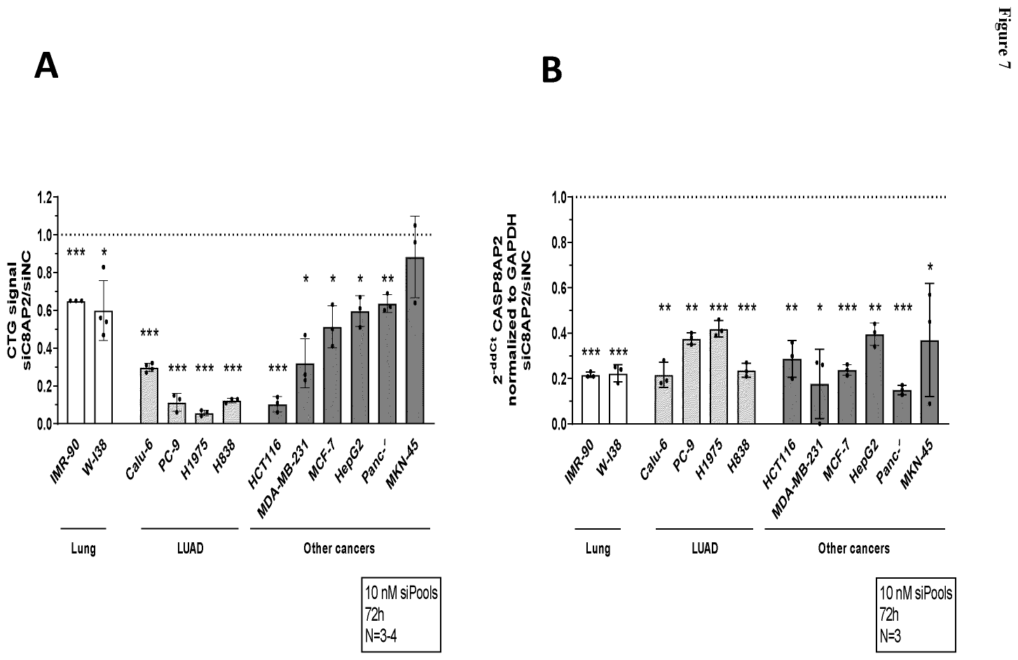

A) CellTiter-Glo cell viability assay (CTG) was performed on six additional non-lung cancer cell lines: HCT116 (colon cancer), MDA-MB-231 (breast cancer), MCF-7 (breast cancer), HepG2 (liver cancer), Panc-1 (pancreatic cancer) and MKN-45 (stomach cancer) at 48 hours post-transfection with siPOOLs against CASP8AP2 (siC8AP2) or negative control siPOOLs (siNC). The siPOOLs were designed and manufactured by siTOOLs Biotech; DNA sequences encoding the sense and antisense strands of the 30 siRNAs of siPool1 are given in SEQ ID NO:20-79; DNA sequences encoding the sense and antisense strands of the 30 siRNAs of siPool2 are given in SEQ ID NO:80-139. HCT116 cells showed a strong decrease in cell viability comparable to the average decrease observed in LUAD cells. MDA-MB-231 cells showed a less pronounced effect comparable to the one observed in the Calu-6 LUAD cell line. Three other cell lines also showed a significant decrease in viability. While MKN-45 cells showed an effect, this did not reach statistical significance (Student's t-test; three asterisks represent p<0.001; two asterisks represent p<0.01; one asterisk represents p<0.05; N=3-4 per condition). The experimental conditions for non-LUAD cancer cell lines recapitulated respective conditions of LUAD experiments described above; see FIG. 7 A.

B) RT-qPCR confirmed significant siPOOLs-mediated CASP8AP2 knockdown in all tested cell lines transfected with siC8AP2 compared to respective cell lines transfected with siNC at 48 hours post-transfection (Student's t-test; three asterisks represent p<0.001; two asterisks represent p<0.01; one asterisk represents p<0.05; N=3 per condition); see FIG. 7 B.

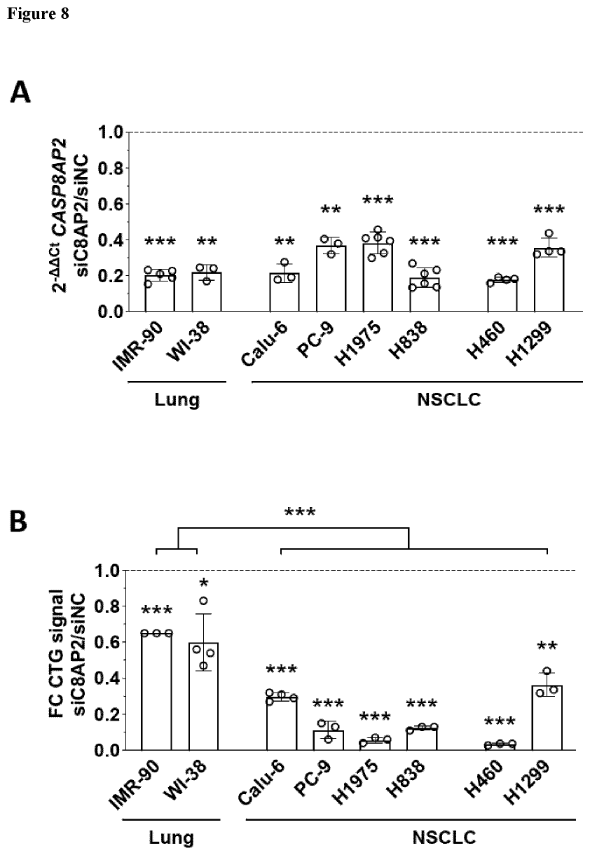

C) RT-qPCR to confirm siPOOL-mediated CASP8AP2 knockdown (siC8AP2, 10 nM) in all tested cell lines transfected with siC8AP2 compared to respective cell lines transfected with non-targeting negative control siPOOL (siNC, 10 nM) at 48 hours post-transfection (n=3-6); see FIG. 8A.

D) CellTiter-Glo cell viability assay (CTG) to assess the effect of CASP8AP2 siPOOL-mediated knockdown (siC8AP2, 10 nM) on non-transformed lung fibroblast cell lines IMR-90 and WI-38, NSCLC LUAD cell lines Calu-6, PC-9, NCI-H1975 (H1975), NCI-H838 (H838), and NSCLC LCLC cell lines NCI-H460 (H460) and NCI-H1299 (H1299) compared to respective cell lines transfected with non-targeting negative control siPOOL (siNC, 10 nM) at 72 hours post-transfection (n=3-4); see FIG. 8B.

For each biological replicate, data were normalized to the respective negative control signal and plotted as individual values. Bar heights represent mean, error bars represent Standard deviation (SD). Significance testing was performed using two-tailed unpaired Student's t-test with Welch's correction, represented as ***, p<0.001; **, p<0.01; *, p<0.05. Significance testing between normal and NSCLC cell lines sensitive to CASP8AP2 silencing was performed using two-tailed unpaired Student's t-test with Welch's correction.

1. A Caspase-8 associated protein-2 (CASP8AP2) antagonist for use in the prevention or treatment of cancer, with the proviso that the CASP8AP2 antagonist is not a peptide comprising the amino acid sequence SMMPDELLTSL (SEQ ID NO: 140) or a peptide comprising the amino acid sequence KLDKNPNQV (SEQ ID NO:141).

2. The CASP8AP2 antagonist for use according to claim 1, wherein the CASP8AP2 antagonist reduces the viability of the cancer cells.

3. The CASP8AP2 antagonist for use according to claim 1, wherein the CASP8AP2 antagonist does not significantly reduce the viability of normal cells in the subject receiving the treatment.

4. The CASP8AP2 antagonist for use according to claim 1, with the proviso that said cancer is not stomach cancer and/or colon cancer and/or rectal cancer.

5. The CASP8AP2 antagonist for use according to claim 1, wherein the cancer is selected from the group consisting of lung cancer, breast cancer, pancreatic cancer or liver cancer; preferably wherein the cancer is a lung adenocarcinoma, squamous cell lung cancer, ER-/PR-positive breast cancer, triple-negative breast cancer, pancreatic ductal adenocarcinoma or hepatocellular carcinoma; more preferably wherein the cancer is a lung adenocarcinoma.

6. The CASP8AP2 antagonist for use according to claim 1, wherein the CASP8AP2 antagonist reduces the transcription and/or translation of the CASP8AP2 gene and/or the stability of the CASP8AP2 mRNA or protein.

7. The CASP8AP2 antagonist for use according to claim 1, wherein the CASP8AP2 antagonist is selected from an antisense oligonucleotide, a small interfering RNA (siRNA), a short hairpin RNA (shRNA), a microRNA (miRNA) or a CRISPR-guide RNA (sgRNA).

8. The CASP8AP2 antagonist for use according to claim 7, wherein the antisense oligonucleotide is capable of binding to and/or is at least partially complementary to a region of the CASP8AP2 gene or regulatory elements in its close vicinity, preferably the CASP8AP2 gene is human.

9. The CASP8AP2 antagonist for use according to claim 7, wherein the siRNA is capable of interfering with the gene expression of the CASP8AP2 gene and comprises a first strand of RNA at least partially complementary to 15 nucleotides of the CASP8AP2 gene, and a second strand of RNA of 15 to 30 nucleotides in length, wherein at least 12 nucleotides of the first strand and second strands are complementary to each other and form a siRNA duplex.

10. The CASP8AP2 antagonist for use according to claim 7, wherein the sgRNA is at least partially complementary to 15 nucleotides of the CASP8AP2 gene and in addition a CRISPR protein lacking endonuclease activity is administered, preferably the CRISPR protein is fused to at least a domain of Kruppel associated box (KRAB) protein.

11. The CASP8AP2 antagonist for use according to claim 1, wherein the CASP8AP2 antagonist is an antibody specifically binding to CASP8AP2 protein, or a binding fragment thereof, preferably wherein the antibody specifically binding to CASP8AP2 protein at least partially blocks the binding of CASP8AP2 to a caspase selected from caspase-3, caspase-7, caspase-8 or caspase-9, preferably the caspase is caspase-8.

12. A pharmaceutical composition comprising a CASP8AP2 antagonist as defined in claim 1 and a pharmaceutically acceptable excipient.

13. A method for identifying a CASP8AP2 antagonist comprising
i) screening a test compound for binding to CASP8AP2 protein;
ii) optionally determining in vitro the caspase-mediated apoptotic activity induced by the test compound identified in step i) in a cancer cell line, wherein the caspase is selected from caspase-3/7, caspase-8 and caspase-9; and
iii) selecting the test compound as a CASP8AP2 antagonist if the test compound binds to CASP8AP2 and optionally increases the apoptotic activity of Caspase-3/7 and/or Caspase-8 and/or Caspase-9 compared to the caspase-mediated apoptotic activity in the absence of the test compound.

14. A method for identifying a CASP8AP2 antagonist comprising
i) screening a test compound for binding to CASP8AP2 protein; and
ii) optionally determining in vitro the cell viability of a cancer cell induced by the test compound identified in step i); and
iii) selecting the test compound as a CASP8AP2 antagonist if the test compound binds to CASP8AP2 and optionally reduces the cell viability of the cancer cell compared to the cell viability of the cancer cell in the absence of the test compound.

15. A method for preparing a pharmaceutical composition comprising a CASP8AP2 antagonist, comprising identifying a CASP8AP2 antagonist by the method of claim 13 and mixing the CASP8AP2 antagonist with at least one pharmaceutically acceptable excipient.
